(19) United States
(12) Patent Application Publication
(10) Pub. No.: US 2007/0117222 A1
(43) Pub. Date: May 24, 2007

(54) Device and Method for Detecting the Presence of an analyte

(75) Inventors: Liberty Sibanda, Harare (ZW); Sara Maria Diana Guido De Saeger, Aalst (BE); Carlos Van Peteghem, De Pinte (BE)

Correspondence Address: BARNES & THORNBURG LLP, P.O. BOX 2786, CHICAGO, IL, 60690-2786, US

(73) Assignee: Universiteit Gent, Gent (BE)

(21) Appl. No.: 11/556,902

(22) Filed: Nov. 6, 2006

Related U.S. Application Data
(63) Continuation-in-part of application No. 10/467,562, filed on Dec. 22, 2003, now abandoned.

(30) Foreign Application Priority Data
Feb. 13, 2003 (EP) 01870025.2
Feb. 13, 2003 (EP) PCT/EP02/01496

(51) Int. Cl. G01N 33/543

(52) U.S. Cl. 436/518

(57) ABSTRACT

The invention relates to a devices and methods for detecting the presence of one or more analytes in an interfering fraction-containing fluid or semi-fluid sample, using an adsorbent medium comprising at least two discrete superposed layers being a first set of one or more clean-up layers and a second set of one or more detection layers through which at least a part of the sample is able to be transported in said order, characterized in that the first set of layers comprises an adsorbent medium capable of adsorbing at least a part of the interfering fraction of the sample and the second set of layers comprises an adsorbent medium containing one or more analyte-receptors capable of retaining the one or more analytes.

### FIELD OF THE INVENTION

The present invention relates to a device and method for detecting the presence of an analyte. It relates in particular to a chromatography screening procedure for assessing toxins, contaminants and clinical compounds frequently encountered in water, food, feed and body fluid samples. More particularly, in the present invention the solid-phase clean-up step and detection of an analyte of interest, e.g. a toxin or contaminant, are carried out simultaneously in one single device.

### BACKGROUND OF THE INVENTION

Our modern environment contains a lot of different substances and some of them are toxic. Type of toxins and other contaminants encountered in the environment are for instance bacterial toxins, mycotoxins, plant toxins, pesticides, hormones and antibiotics. Some toxins and contaminants are very stable and produce severe illness when ingested, inhaled, or introduced into the body by any other means. For instance, mycotoxins are known to be poisonous, mutagenic, teratogenic or carcinogenic when consumed by humans or animals. Mycotoxins are secondary metabolites of low molecular weight produced by molds and fungi during their growth on food and feed. Mycotoxins may remain in food and feed long after the mold or fungus that produced them has died. Therefore products that are not visibly moldy or do not test positive for mold count can still contain potentially dangerous levels of mycotoxins. Diseases caused by mycotoxins in humans and animals are called mycotoxicosis and are specific to the mold species and the toxin produced. Several types of mycotoxins exist, such as aflatoxins, ochratoxins, vomitoxins, fumonisins, T-2 toxin, patulin, zearalenone . . .

Several countries have currently established or proposed regulations for control of mycotoxins (primarily the aflatoxins) in food and animal feed. In order to harmonize these regulations, the Food and Drug Administration has established guidelines for the levels of aflatoxin permitted in commodities for further processing. The permitted levels vary depending upon the intended end usage of the commodity. Many countries have also established regulations for ochratoxin A (OA), trichothecenes, zearalenone, patulin and fumonisins. Maximum tolerated levels for OA range from 1 to 50 μg/kg for food and from 100 to 1000 μg/kg for animal feed.

It is obvious that the enforcement of these regulations require accurate monitoring of suspected commodities. Therefore, there is a continuous need for a very simple, rapid and inexpensive method for detecting mycotoxins.

The same applies for hormones, pesticides and antibiotics, which are often encountered in our food supply. For instance, in many situations it is of vital importance to be able to detect the presence of small amounts of antibiotics. This is the case in food industries where the increased use of antibiotics and chemotherapeutic substances in the treatment of animals has created a need for a simple, reliable and sensitive method of determination.

Many analytical methods exist in prior art for toxins, mycotoxins and other contaminants in food and feed. In general, most methods used are related to the separation and detection of analytes in a test sample using a two-steps procedure. In a first step the test sample is cleaned-up and followed by a second suitable detection step.

To date, solid phase clean-up systems are used for isolating the molecule of interest by allowing it to bind to the bonded stationary phase. Next, the unbound compounds are washed away and out of the column. The compound of interest is eluted using an appropriate buffer capable of dislodging the adsorbed molecule from the stationary phase. The eluate is evaporated to dryness and the residue re-dissolved in a smaller volume to pre-concentrate it in order to carry-out analyses such as enzyme-linked immunosorbent assay (ELISA), radio immunoassay (RIA), high performance liquid chromatography (HPLC), liquid chromatography mass spectrometry (LC-MS) and gas chromatography mass spectrometry (GC-MS). Several prior art patent and patent applications are concerned with said methods.

WO 89/03037 and U.S. Pat. No. 5,178,832 relate to a method and testing column for the selective immobilization and detection of mycotoxins in solution. It has been discovered that certain minerals, particularly various naturally occurring forms of Aluminum oxide, will preferentially bind selective mycotoxins from a mixture of mycotoxins. These adsorbents, when used in various combinations and/or in conjunction with the adsorbents of the prior art, permit the construction of detector tubes which can resolve mycotoxins in solutions and provide a semi-quantitative fluorescent determination of their concentration in feed or foodstuff samples. The detector tubes comprises transparent tubes packed with isolated layers of selected minerals. A solvent extract from a sample potentially contaminated with mycotoxins is passed through the column. As the mycotoxin mixture passes through the detector tube and is contacted by the various mineral adsorbants, selected mycotoxins are immobilized on a specific mineral while other mycotoxins and co-extracted organic compounds pass through that layer to be immobilized on subsequent downstream mineral layers. The presence of mycotoxins is determined by examining the developed detector tube under a long wave uv light source.

U.S. Pat. No. 5,110,558 relates to a method and apparatus for adsorption and detection of analytes. The method and apparatus can be employed in the field for rapid adsorption of analytes and is particularly useful for detection of mycotoxins. A sample to be analyzed is prepared in solution and placed in a test tube. A tube-like adsorption column having a seal and a valve member is forcefully fed into the test tube to force solutions through the valve member into the column and through a filter and adsorbent to trap interferences. The semi-purified solution may then be analyzed for the presence of analytes. The column with the purified solution may be further employed with a second smaller adsorption column similarly equipped with a seal and valve member fitting within the first column. In similar fashion the second column may be forced into the first column to expel the solution therein into the second column and through one or more selective adsorbents for different analytes such as one or more mycotoxins. Detection of the adsorbed analyte may be made by shining a fluorescent or “black” light on the adsorbent which fluoresces to indicate presence of the analyte.

However, all these prior art analytical methods have several disadvantages. Most prior art methods are time consuming and expensive. This applies in particular for chromatographic procedures. It takes several hours to several days to complete a chromatographic analysis. In addition, extensive clean-up is often required before a sample can be applied, for example, on a HPLC column. Moreover, these techniques are not well suited for performing analyses in the field or away from a laboratory in as much as they require complex instruments and a relatively high degree of skill on the part of the person performing the analysis.

Many ELISA screening kits have also been introduced in recent years. However, sophisticated equipment and qualified personnel are still needed to perform ELISA's, and their application is restricted to laboratories.

WO99/676447 describes a multi-layer testing column comprising a plurality of membrane layers vertically stacked within the chamber of the column and include at least a plurality of solid-phase substrates each carrying a different anti-analyte. Some of the uppermost and lowermost layers are preferably filter layers, which substantially prevent passage of large particles, e.g. blood cells to other membrane layers. A sample can be placed in the chamber such that specific analytes of the sample are bound to the anti-analytes. A sensor can be located within the housing to receive a signal from the substrates and to generate a corresponding electric signal.

However, a need exists for rapid and convenient tests for analyte detection. In particular, such assays need to be simple and easy to use when performed in the field and interpreted by non-technical users. For instance, mycotoxin production occurs mostly during the harvest period after cereals, oilseeds or nuts have begun to dry, before they attain the moisture level best suited for storage. Storage of the foodstuffs under proper temperature and humidity conditions will prevent further contamination. Thus, it is important that contaminated lots are detected as early, in the food processing chain, as possible.

Therefore, the principal object of the present invention is to provide a binding device and assay method for detecting analyte contamination for use in the field. Moreover, the devices and methods of the present invention are easy to handle, inexpensive, provide rapid and reliable results, and adaptable for field testing.

### SUMMARY OF THE INVENTION

In the present invention, devices and methods are disclosed for detecting the presence or absence of one or more analytes in fluid or semi-fluid sample containing an interfering fraction.

According to a first embodiment, the device of the invention comprises:
(a) a transparent housing,
(b) inlet means for the sample to be analyzed,
(c) outlet means, and
(d) at least two discrete superposed layers being one or more layers for removing the interfering fraction from the sample and one or more layers for detecting the one or more analytes. Typically, the layers are arranged such that the device comprises a first set of one or more cleaning-up layers and a second set of one or more detection layers through which at least a part of the sample is able to be transported in said order, characterized in that at least one layer of the first set of layers comprises an adsorbent medium capable of actively adsorbing at least a part of the interfering fraction of the sample and at least one layer of the second set of layers comprises an adsorbent medium containing an analyte-receptor capable of specifically retaining one of the analytes.

According to a further specific embodiment, the analyte-receptor present on the adsorbent medium of the at least one layer of the second set of layers is a protein which specifically binds an analyte of interest, more particularly an antibody specifically recognizing one of the analytes of interest in the sample under investigation.

The above-disclosed device has the unique feature of the ability to trap interferences and detect analytes in one single step. Said analytes are, for example, toxins, mycotoxins, pesticides, drugs, antibiotics or hormones present in water, food, feed or body fluid samples. According to one embodiment, the adsorbent medium of at least one layer of the first set of layers is selected from the group consisting of agarose, silica, sepharose, dextrans or derivatized versions thereof. The adsorbent medium of the first set of layers is selected so as to ensure optimal retention of the interfering fraction of the sample to be analyzed. According to a particular embodiment, the adsorbent medium of the one or more first layers is characterized in that at least part of the adsorbent medium comprises a derivatized surface, ensuring reactivity with the relevant interfering fraction of the sample. More particularly, the surface is derivatised with a functional group selected from the group consisting of trimethylaminopropyl, n-propyl-ethylene-diamine (PSA), octadecyl (18), Diol (2OH) and cyanopropyl (CN) or aminopropyl (NH₂) groups. In a particular embodiment, an aminopropyl derivatized surface is used. The adsorbent medium of at least one of the second set of layers comprises an analyte receptor, determined in function of the analyte to be detected. In particular embodiments, the adsorbent medium of the second layer similarly comprise components such as agarose, silica, sepharose, or dextrans. These components can be derivatized for covalent binding of the analyte receptor in the generation of the second layer. However, the adsorbent medium of the second set of layers no longer contains active functional groups when ready for use in the methods of the present invention.

According to a further embodiment the housing of the device of the invention is tubular. Furthermore, the inlet means of the device of the invention may be connectable to pressure means, for instance a hand-held portable pressure means to keep with field applications. Such pressure means are capable of exerting pressure upon said sample to force the transport of the sample from the inlet means to the outlet means. For example, the housing of the device of the invention can consist of a syringe and the pressure means of a syringe plunger.

The invention further relates to methods for detecting the presence or absence of one or more analytes in a fluid or semi-fluid sample containing an interfering fraction, the methods comprising the steps of:
(a) applying the sample in a flow-through motion onto an adsorbent medium comprising at least two sets, each of one or more layers superposed such as to define at least a first and a second set of layers in which the first set of layers is capable of actively adsorbing at least a part of the interfering fraction of the sample without retaining specifically the analyte, and whereby the second set of layers is capable of specifically retaining the analyte(s) of interest present in the sample,
(b) optionally, washing the adsorbent medium in order to remove possible color interference of the second set of layers,
(c) optionally, applying a predetermined amount of one or more binder molecules onto the adsorbent medium, each of the one or more binder molecules capable of being retained specifically by one layer of the second set of layers, and able to provide detection of the presence or absence of the corresponding analyte of interest in the one layer of the second set of layers,
(d) finally, detecting the presence or absence of said analyte(s) specifically retained in the one or more layers of the second set of layers.

In particular embodiments of the methods of the invention, at least part of the adsorbent medium of at least one of the second set of layers, and optionally of each of the second set of layers comprises an analyte-receptor. More particularly the analyte receptor is a protein capable of specifically binding one of the one or more analytes, the protein corresponding to one of the set of antibody-antigen pair, receptor-ligand pair, enzyme-substrate pair, etc. According to a specific embodiment, the analyte receptor is an antibody specifically recognising one of the one or more analytes in the sample.

In particular embodiments of the methods of the invention, at least part of the adsorbent medium of at least one of the first set of layers comprises a derivatized surface. Most particularly, the derivatised surface is derivatised with a functional group selected from the group consisting of trimethylaminopropyl, n-propyl-ethylene-diamine (PSA), octadecyl (18), Diol (2OH) and cyanopropyl (CN) or aminopropyl (NH₂) groups. In a particular embodiment an aminopropyl derivatized surface is used.

As an optional feature of the methods of the present invention, the predetermined amount of binder molecule is labeled with an enzyme or a bioluminescent, chemiluminescent, phosphorescent or fluorescent molecule.

According to one embodiment of the methods according to the present invention, where the binder molecule is an enzyme, the method further comprises the following step after step (c) and before step (d): applying a substrate of the enzyme onto the adsorbent medium, the substrate being capable of reacting with the binder molecule and being capable of generating a detectable signal. According to further particular embodiments, the methods according to the present invention further comprise washing the two sets of layers of the adsorbent medium in order to remove all unbound binder molecule from the second layer before applying the substrate onto the absorbent medium. Optionally, the predetermined amount of a binder molecule may be a predetermined amount of a labeled analyte, which, by competing with the analyte in the sample for the corresponding analyte receptor, allows detection of the absence or presence of the analyte of interest in the second layer.

Accordingly, as yet another optional feature, more particularly where the label of the labeled analyte is an enzyme, the method according to the present invention further comprise after step (c) and before step (d) applying a substrate onto the adsorbent medium, the substrate being capable of reacting with the labeled analyte molecule and being capable of generating a detectable signal.

According to one embodiment of the methods of the present invention, the adsorbent medium comprises one first clean-up layer and one second detection layer and the methods comprise detecting the analyte in the one second layer.

According to another embodiment, the adsorbent medium comprises one first clean-up layer and two or more second detection layers, each of which capable of specifically binding an analyte, and the methods comprise detecting different analytes in the two or more second layers. Most particularly, each of said second layer is capable of detecting a different analyte. In a specific embodiment, two or more of said second layers is capable of detecting the same analyte, optionally using a different analyte-receptor.

According to particular embodiments, the methods of the invention comprise a pre-treatment step, which pre-treatment step comprises extracting, concentrating or dissolving the analyte of interest in the sample under investigation with a pretreatment solvent. The nature of the pretreatment solvent is determined by the nature of the analyte receptor and the analyte and is selected such that, upon application to the adsorbent medium, it does not denature the analyte receptors of the second set of layers.

According to a further particular embodiment, the methods of the present invention comprise the step of pre-treating the sample with a pretreatment solvent. According to one embodiment, the pretreatment solvent comprises a high percentage (more than 30%) of organic solvent. However, where the analyte receptor of the one or more second layers is proteinaceous, the methods of the present invention envisage the application of the sample onto the adsorbent medium in a solvent which comprises between 0 and 30% of organic solvent and between 70 and 100% of an aqueous solvent. Accordingly, in one embodiment, the methods of the present invention include a dilution step, whereby the pretreatment solvent is diluted so as to obtain a solvent comprising between 0-30% organic solvent and between 70-100% aqueous solvent.

The invention further relates to methods for detecting the presence or absence of one or more analytes in a fluid or semi-fluid sample containing an interfering fraction, the methods comprising the steps of:
(a) applying the sample to a device comprising:
   (i) a transparent housing,
   (ii) inlet means for the sample,
   (iii) outlet means for the sample, and
   (iv) an adsorbent medium comprising at least two discrete sets of layers superposed such as to define at least a first and a second set of layers in which the first set of layers actively adsorbs at least part of the interfering fraction in the sample without retaining specifically the analyte(s) and whereby each layer(s) of the second set of layers specifically retains the one or more analyte(s),
   whereby the sample is applied to the device via the inlet means; and
(b) detecting the presence or absence of the one or more analytes retained in the layer(s) of the second set of layers.

In a particular embodiment, the invention provides methods for detecting the presence or absence of an analyte in a fluid or semi-fluid sample containing an interfering fraction, the method comprising the steps of:
(a) applying said sample to a device comprising:
   (i) a transparent housing,
   (ii) inlet means for said sample,
   (iii) outlet means for said sample, and
   (iv) an adsorbent medium comprising at least two discrete layers superposed such as to define at least a first and a second layer in which the first layer comprises a derivatised surface and the second layer comprises an analyte receptor, wherein the first layer actively adsorbs at least part of the interfering fraction in the sample without retaining specifically the analyte and whereby the second layer specifically retains the analyte,
   whereby the sample is applied to said device via said inlet means; and
(b) detecting the presence or absence of the analyte retained in the second layer.

As an optional feature to this particular embodiment, the adsorbent medium may further comprise one or more additional layers, each capable of specifically retaining a different further analyte present in the sample, which method comprises detecting the presence or the absence of the further analytes retained in the one or more additional layers.

As another optional feature, the sample may be applied onto the adsorbent medium of the device as described above by a pressure means capable of exerting pressure upon the sample to force the transport of the sample from the inlet means to the outlet means. Optionally, the housing of the device consists of a syringe and the pressure means of a syringe plunger.

As yet another optional feature, at least one layer of the first set of layers comprises a solid support material selected from the group consisting of agarose, silica, sepharose or dextrans and wherein at least part of the surface of this solid support material is derivatized to produce a bonded matrix.

According to a particular embodiment, the derivatized surface of the first layer is derivatized with aminopropyl groups.

According to a particular embodiment, the detection of the presence or absence of the one or more analytes of interest in the second layer in the methods of the invention is done visually or by suitable detector means.

Finally, the present invention also relates to a kit consisting of at least one of the devices of the invention as described above and one or more of the following:
(a) a pretreatment solvent capable of extracting, concentrating or dissolving the analyte of interest in the sample under investigation,
(b) pressure means connectable to the inlet means of the device and capable of forcefully exerting pressure upon the sample under investigation, to force at least part of the sample from the inlet to the outlet means of said device,
(c) a washing solution capable of removing possible color interferences of the second layer,
(d) one or more binders, each capable of interacting with the corresponding non-occupied analyte-receptor(s) of the second set of layer(s), and able to provide detection of the presence or absence of said the corresponding analyte(s) of interest in the second layer(s),
(e) one or more labeled binder molecules capable of interacting with the corresponding non-occupied analyte-receptor(s) of the second layer(s),
(f) a labeled derivative of each of the analyte molecules under investigation,
(g) a washing solution capable of removing all unbound binder from the second layer(s), and
(h) a substrate solution capable of reacting with the labeled binder molecule(s) thereby generating a detectable signal.

The nature of the pretreatment solvent is determined by the nature of the analyte receptor and the analyte and is selected such that it does not denature the analyte receptors of the second set of layers of the adsorbent medium.

According to one embodiment, the pretreatment solvent comprises a high concentration of organic solvent (i.e. more than 30%). According to a further specific embodiment, the analyte receptor is a protein and the methods of the invention further comprises diluting thepretreatment solvent comprising a high concentration of organic solvent (i.e. more than 30%) to a concentration of between 0 and 30% of organic solvent and between 70 and 100% of an aqueous solvent, for application to the adsorbent medium.

The devices, methods and kits of the invention permit rapid screening of important analytes, such as, but not limited to, environmental contaminants like pesticides, food toxins and mycotoxins, antibiotics, therapeutic drugs and hormones and their respective conjugates, metabolites and derivatives.

Among the advantages which may be realized by the use of the devices and methods which embody the present invention are: speed of analysis (test takes approximately 15 minutes for semi-quantitative results and more than one analyte may be detected in one test), ease of use (technical expertise is not required), sensitivity, economy (minimal production costs), stability (no refrigeration is required) and flexibility (the device and associated method provide a ready-to-go field test).

The embodiments set out above and other features and additional advantages of the present invention are more fully set forth in the following detailed description below and the accompanying figure and examples.

### BRIEF DESCRIPTION OF THE FIGURES

FIG. 1. Schematical cross-sectional view of a device according to an embodiment of the invention.

FIG. 2. A chromatographic profile of a roasted coffee sample spiked with OA and analyzed without an aminopropyl solid-phase clean-up step. The y-axis represents responses of the recorder (in peak area units) to the fluorescence detector signal.

FIG. 3. Chromatographic analysis of an OA-spiked (10 ng/g) roasted coffee sample by HPLC after aminopropyl solid-phase clean-up, according to one embodiment of the invention. The y-axis represents recorder responses to fluorescence detector signal.

FIG. 4. The effect of methanol concentration on the retention of OA by the aminopropyl column, according to an embodiment of the invention.

FIG. 5. Schematical cross-section view of a device according to an embodiment of the invention comprising two second layers.

FIG. 6. Schematical cross-section view of a device according to an embodiment of the invention comprising two detection layers.

### DETAILED DESCRIPTION OF THE INVENTION

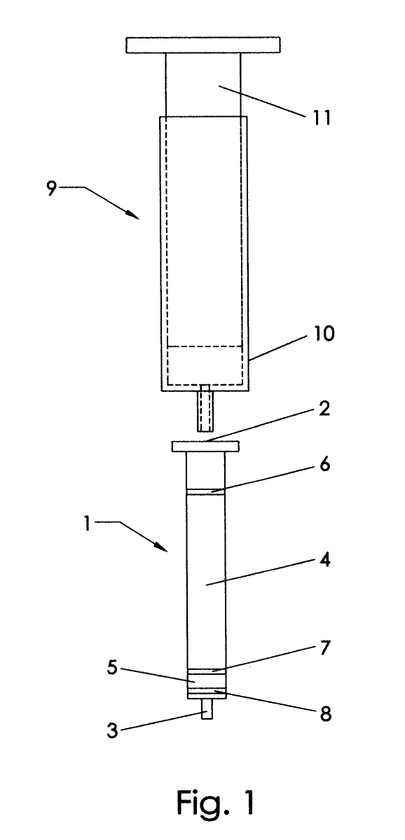

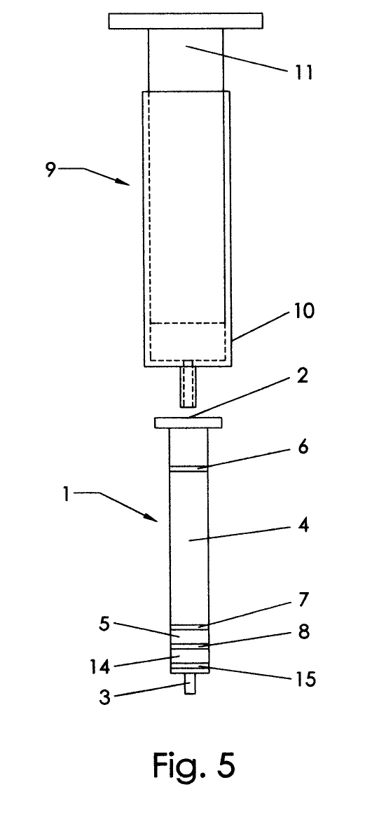

The main embodiments of the invention, and several variations of these embodiments, will be described with reference to FIG. 1 or FIG. 5. Other embodiments will be apparent to those skilled in the art.

According to a first embodiment the present invention relates to a device for detecting the presence or absence of one or more analyte(s) in an interfering fraction-containing fluid or semi-fluid sample, said device comprising:
(a) a transparent housing,
(b) inlet means for the sample to be analyzed,
(c) outlet means for the sample, and
(d) at least two discrete superposed layers, comprising a first and a second set of one or more layers through which at least a part of the sample is able to be transported in said order, in which said first set of one or more layers comprises an adsorbent medium comprising a derivatized surface capable of actively adsorbing at least a part of the interfering fraction of the sample and in which at least one of the second set of one or more layers comprise an adsorbent medium containing an analyte-receptor, for instance an antibody capable of specifically retaining or recognizing an analyte.

Optionally, the inlet means of the device are connectable to a pressure means capable of exerting pressure upon the sample to force the transport of the sample from the inlet means to the outlet means.

Optionally, the outlet or inlet means of the device are connectable to vacuum or suction means capable of exerting pressure on the sample in the device to force transport of the sample from the inlet means to the outlet means.

In a further embodiment of the devices of the invention, the pressure or vacuum means comprises a syringe plunger and the transparent housing is a syringe.

In a further embodiment of the devices of the invention, the pressure means is a syringe plunger, the transparent housing is a syringe, the inlet means is the opening of the syringe barrel receiving the syringe plunger (or an opening in the barrel beneath between the opening of the syringe plunger and the adsorbent medium) and the outlet means is one or more openings of the barrel on the other side of the adsorbent medium from the syringe plunger.

In a further embodiment, in the devices of the invention, the vacuum means is a syringe plunger, the transparent housing is a syringe, the inlet means is one or more openings of the syringe barrel on the opposite side of the adsorbent medium from the syringe plunger and the outlet means is the opening of the syringe plunger.

FIG. 1 depicts one embodiment of the devices for use in the present invention consisting of two superposed layers within the housing. These devices comprise a transparent housing, in particular a tube (1), inlet (2) and outlet (3) means, two superposed layers within the housing whereby the first layer, which is a clean-up layer (4) comprises an adsorbent material capable of adsorbing at least part of the interfering fraction of the sample, and the second layer, which is a detection layer (5) comprises an adsorbent material containing an analyte-receptor capable of specifically retaining the analyte. The device optionally comprises three grids, a first grid (6) is provided above the first layer (i.e. the clean-up layer) (4), a second grid (7) in between the clean-up layer (4) and the second layer (i.e. a detection layer) (5) and a third grid (8) beneath the second layer (5), providing for a physical separation barrier in-between the layers. A pressure means (9) consisting of a syringe barrel (10) operated with a plunger (11) is used to force the sample under investigation from the inlet means to the outlet means.

According to a particular embodiment, the device of the invention comprises a first clean-up layer and two second layers which are detection layers. Accordingly, in one embodiment, the devices of the present invention consist of three superposed layers within the housing. Most particularly, the devices correspond to the device illustrated in FIG. 5 and comprises a transparent housing, in particular a tube (1), inlet (2) and outlet (3) means, three superposed layers within the housing whereby the first layer, which is a clean-up layer (4) comprises an adsorbent material capable of adsorbing at least part of the interfering fraction of the sample, and two second layers, which are detection layers (5, 14) comprise an adsorbent material containing an analyte-receptor capable of specifically retaining a first analyte and a second analyte, respectively. The devices according to this embodiment optionally comprise four grids, a first grid (6) is provided above the first layer (i.e. the clean-up layer) (4), a second grid (7) in between the clean-up layer (4) and the first of the two second layers (5), a third grid (8) between the first of the two detection layers (5) and the second of the two detection layers (14) and a fourth grid (15) beneath the second of the two detection layers (14), providing for a physical separation barrier in-between the layers. In the embodiment illustrated in FIG. 5, a pressure means (9) consisting of a syringe barrel (10) operated with a plunger (11) is used to force the sample under investigation from the inlet means to the outlet means.

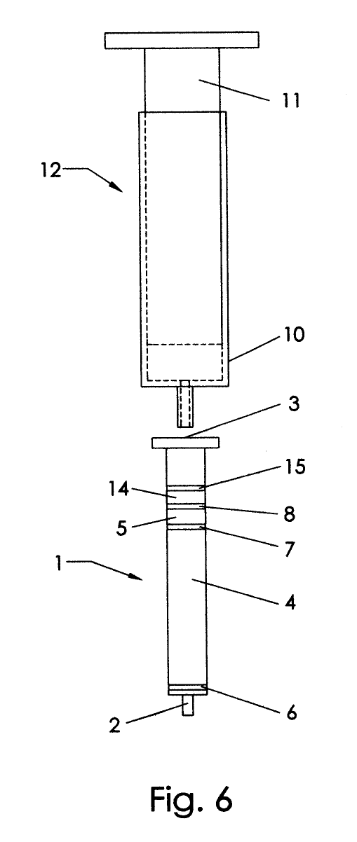

An alternative embodiment of the devices of the present invention comprises a pressure means which is a vacuum means for aspiring the sample from the inlet means to the outlet means, as illustrated in FIG. 6 for a device comprising one clean-up and two detection layers. In this embodiment, the device comprises a transparent housing, in particular a tube (1), inlet (2) and outlet (3) means, three superposed layers within the housing whereby the first layer is a clean-up layer (4) which comprises an adsorbent material capable of adsorbing at least part of the interfering fraction of the sample, the second layer is a detection layer (5) which comprises an adsorbent material containing an analyte-receptor capable of specifically retaining a first analyte, and the third layer is another detection layer (14) which comprises an adsorbent material containing an analyte-receptor capable of specifically retaining a second analyte. The device optionally comprises four grids, a first grid (6) is provided beneath the first layer (i.e. the clean-up layer) (4), a second grid (7) in between the clean-up layer (4) and the second layer (i.e. a detection layer) (5), a third grid (8) between the second layer (5) and the third layer (i.e. another detection layer) (14) and a fourth grid (15) above the third layer (14), providing for a physical separation barrier in-between the layers. A vacuum means (12) consisting of a syringe barrel (10) operated with a plunger (11) is used to force the sample under investigation from the inlet means to the outlet means.

The invention further relates to the use of any of the devices of the invention for detecting the presence or absence of one or more analytes in an interfering fraction-containing fluid or semi-fluid sample under investigation. In the rest of this description, a system comprising only two layers, i.e. a first layer which is a clean-up layer and a second layer which is a detection layer will usually be described but it will be well understood by the person skilled in the art that all what is described hereunder can similarly be applied to a system comprising two or more detection layers to detect one or more, more particularly two or more analytes. Accordingly, in what follows, the ‘first layer’ is clean-up layer and the ‘second layer’ is the detection layer. However, the features below equally apply to a system comprising a first set of one or more clean-up layers and a second set of one or more detection layers.

In particular embodiments of the methods of the invention, the detection of the presence or absence of the analyte of interest in the second layer is done visually and is for instance based on whether a color develops or not.

In specific embodiments of the methods making use of the device of the present invention, a sample, containing an analyte of interest to be screened and an interfering fraction, is applied via the inlet means (2) of the device onto the two superposed adsorbent layers. The nature of the analyte of interest is not critical. In particular embodiments, the analyte of interest can be selected from the group consisting of toxins, mycotoxins, pesticides, drugs, antibiotics, hormones or one of their respective conjugates and derivatives. A list of possible analytes which can be screened by this invention are listed in Table 1 (not exhaustive).

As stated above, the invention provides for at least two superposed adsorbent layers comprising a first (4) and a second (5) layer through which at least a part of the sample under investigation is able to be transported in said order.

The first layer (4), which is the clean-up layer in the devices and methods of the present invention, comprises an adsorbent medium capable of actively adsorbing at least a part of the interfering fraction of the sample under investigation.

According to one embodiment, this is achieved by derivatizing the surface of the solid support material to introduce specific chemical groups which confer a particular solid phase/matrix interference interaction. The derivatization of the solid support surface produces what is known in solid phase extraction (SPE) as a bonded matrix or bonded solid support. The solid support usually used for solid phase extraction are agarose, silica, sepharose and dextrans, including derivatized silica.

The expressions “solid phase”, “solid support phase” and “solid support material” are herein used interchangeable and relate to the material which is used as adsorbent medium.

Contrary to classical solid phase extraction procedures however, the solid phase of the first layer in the device and methods of the present invention, is used to actively adsorb the interferences or interfering fraction present in the sample, rather than the analyte(s).

Thus, the expression “actively adsorbing” means that the first layer is for instance a stationary solid phase used for cleaning-up the sample, and is made-up of an adsorbent medium comprising at least partly derivatized solid support material which adsorbs targeted interferences by means of non-specific interactions (Van der Waal's), non-polar, polar or ionic interactions. One example of a derivatizing molecule is for instance carbon, to form e.g. Si—O—C₁₈Hₙ. Other examples are described below.

Bonded silica supports or bonded silica sorbents are prepared by reaction of the surface hydroxyl groups (silanols) with halo- or alkoxysilyl derivatives, resulting in the covalent bonding of a wide range of functional groups. To give the solid support, for instance the silica support the desired properties for a particular adsorption, extraction or separation, an organic moiety is attached to the solid support, e.g. the silica. The solvated bonded solid supports offer through the organic moiety an array of chemical environments which can be selected for specificities suitable for the interference of each sample. The expression “solvated” as used herein relates to a state wherein a bonded solid phase interacts with a solvent whereby the derivatizing compound on the surfaces interacts with the liquid in such a way as though it was in solution.

Bonded silica solid supports exhibit unusual physical stability. They do not shrink or swell in contact with aqueous or organic solvents. The bonded silica solid phase particles are rigid and will tolerate a high viscosity flow of samples and solvents when these materials are packed into small extraction columns.

According to a further embodiment the invention relates to devices and methods of detection as described above wherein said solid phase of the first layer adsorbs interferences in at least one of three different ways namely non-polar, polar and ionic interactions. Non-polar interactions are those based on the dispersion forces (van der Waal's forces) that occur between the carbonaceous component of the interference in the sample and the functional group of the derivatized solid support surface. Van der Waal's forces of attraction are non-bonding interactions and are only a function of the surface area of the inter-molecular contact. The principal non-polar chemical groups used for derivatization are those with the C₁₈, C₈, C₂, cyclohexyl and phenyl groups whose long carbon chains offer a large surface area for inter-molecular interaction. For polar interactions various sorbent phases are used including aminopropyl, cyanopropyl, diol, N-propyl-ethylene-diamine. Hydrogen bonding interactions are the polar interactions most widely used. Hydroxyl and amino groups are the common hydrogen bond donors and these typically interact with other groups containing oxygen, nitrogen and sulphur atoms. The third form of derivatization provides ionic-based interactions. This principle is based on the attraction of positively or negatively charged compounds to adsorb onto the stationary bonded phase. Bonded chemical groups for ionic interactions include diethylaminopropyl, trimethylaminopropyl, benzenesufonylpropyl, sulfonypropyl, carboxymethyl and the weekly ionic aminopropyl, and N-propylethylene-diamine (PSA). The solvated form of ionic matrices or the derivatized solid supports offers one or more charged groups (positive or negative) to which an interference with an opposite charge will bind.

Therefore, according to a further embodiment, the invention relates to devices and methods of detection as described herein, wherein the surface of the adsorbent solid support of the first layer of the adsorbent medium comprises at least one of the following functional, chemical groups: octadecyl, octyl, ethyl, cyclohexyl, phenyl, aminopropyl, cyanopropyl, diol, n-propyl-ethylene-diamine, diethylaminopropyl, trimethylaminopropyl, benzenesulfonylpropyl, sulfonylpropyl and carboxymethyl. It should be obvious to the man skilled in the art, that the modifications or functional groups which can be displayed by the adsorbent medium are not restricted to the above list, which is merely given to provide examples. For instance, as explained above, alkyl groups containing long carbon chains, such as from C₈ to C₁₈ or even longer are also envisaged as possible functional groups.

In the methods of the present invention the first layer solid phase clean-up step is used for trapping compounds in samples which may otherwise interfere with subsequent analysis steps, and are therefore referred to as ‘interferences’ or generally the ‘interfering fraction’ of the sample. For instance, the interferences may influence capturing of an analyte of interest on the second adsorbent layer of the adsorbent medium. Additionally, said interferences may also interfere with the subsequent detection reaction. Additionally or alternatively, such interferences may generally hamper the purification and/or detection of an analyte of interest in the sample, e.g. an interference with an identical retention time on HPLC as the analyte of interest, can hamper HPLC purification of the analyte. Samples with interfering matrices include, but are not limited to, samples of food, feed, industrial waste-water, urine, and blood. The main principle is to use the stationary bonded phase of the first layer to adsorb and trap interferences while the analyte remains dissolved in the mobile phase and is subsequently adsorbed by the second layer of the adsorbent medium or device. Accordingly, the adsorbing and trapping of interferences in the first layer is typically based on non-specific interaction(s) of the interfering fraction with the adsorbent medium of the first layer. The ‘clean-up layer(s)’ of the devices of the present invention will thus comprise adsorbent medium which is, prior to use in the methods of the present invention not linked to proteins such as analyte receptors or other targeting molecules such as antibodies or receptor ligands. Accordingly, the clean up layer(s) is(are) typically a derivatized solid support material free of analyte receptors.

The device and methods of the present invention are characterized by the feature that the reagents used are compatible with both the clean-up and assay part, whereas this is not the case in conventional solid-phase extraction methods. More particularly, in order to allow combined clean-up and analyte detection, the devices and methods of the present invention make use of a clean-up and detection layer, which are selected such that they are compatible, i.e. that the solvents required are compatible with both clean-up and detection. This is discussed more in detail below.

According to a particular embodiment, the methods of the present invention further comprise a pretreatment step. Indeed, to effectively increase the sensitivity of analyte-detection, the sample is often pretreated by dissolving or extracting it with a specific solvent prior to applying said sample onto the adsorbent medium. Additionally or alternatively, the sample is non-fluid and extraction is required to allow detection. Accordingly, such pretreatment may extract, concentrate or dissolve the analyte from the sample. For instance, a solven is used which creates an environment most favorable to the analyte. This decreases the solid/mobile phase partition co-efficient in favor of the mobile phase. The analyte is then directly eluted as the sample is applied through the first layer of the adsorbent medium or device. The interfering fraction of the sample is retained on the solid phase of the first layer by the specific modes of interactions provided for by the chemical environment of the first layer.

According to the present invention, the sample is transported from the first layer to the second layer of adsorbent medium, where the analyte is specifically retained. Typically, this is achieved by the presence, in the second layer, of an analyte-receptor. The term “analyte-receptor” as used herein refers to a molecule capable of specifically binding the analyte. The nature of the analyte-receptor may vary and includes, but is not limited to, a protein, a peptide, an antibody, a metal ion, a lectin, a carbohydrate, etc . . . In a particular embodiment of the methods and devices of the present invention, the analyte-receptor is a proteinaceous compound which interacts with the analyte as an antibody-antigen pair, a receptor-ligand pair, an enzyme-substrate pair, etc. Thus, the analyte-receptor may be an antibody, other protein, peptide or peptide fragment, binding moiety or other binding partner such as a receptor or fragment thereof specifically recognizing or binding to the analyte. According to a specific embodiment, the analyte-receptor is an antibody specifically recognising the analyte in the sample.

Thus, according to a particular embodiment of the present invention, the second layer (5) (or sets of layers) of the adsorbent medium or device comprises an adsorbent medium containing an analyte-receptor capable of retaining the analyte(s) of interest. The adsorbent medium is a solid support material onto which one or more specific analyte-receptor(s) is/are present. Typically, the analyte-receptor is covalently bound to the solid support material. In a particular embodiment, the solid support of the second layer is one which supports specific protein interactions, such as immunological reactions or receptor-ligand interactions. For instance, agarose, sepharose and dextrans are solid supports used in immunological and immunoaffinity solid phases. The analyte-receptor can be bound to the adsorbent medium of the second layer by derivatization of the solid support material. In one embodiment, the analyte-receptor is a protein or another molecule with a primary amine group and the solid support medium is CNBr-activated Sepharose.

Sepharose is bead-formed agarose gel which displays all the features required for a successful immobilization of biologically active molecules. The hydroxyl groups on the sugar residues are easily derivatized for covalent attachment of an analyte-receptor. The open-pore structure and the exclusion limit of Sepharose 4B in gel filtration (MW 20×106) makes the interior of the matrix available for analyte receptor attachment and ensures good binding capacities. Sepharose 4B exhibits extremely low non-specific adsorption. Adsorbents based on Sepharose are stable under a wide range of experimental conditions such as high and low pH, detergents and dissociating agents. CNBr-activated Sepharose 4B enables analyte receptors containing primary amino groups to be safely, easily and rapidly immobilized by a spontaneous reaction.

The present invention provides for an adsorbent medium comprising a combination of clean-up and detection layers, such that removal of one or more interfering fractions and detection of one or more analytes can be performed in one step. This requires that the nature of the adsorbent medium of the first layer be selected in function of the interfering fraction and the analyte such that the first and second layers be compatible, i.e. that the reagents/pretreatment of the sample required for the first (clean-up) layer be compatible with the reagents/pretreatment of the sample required for the second (detection) layer. Additionally, it requires that the breakthrough volume of the first layer is significantly higher for the interfering fraction in the sample than for the analyte, so as to ensure appropriate removal of the interfering fraction from the analyte in the first layer.

The breakthrough volume is defined as the sample volume eluted from the outlet means until analyte concentration reaches 1% of the analyte concentration added at the inlet means. The breakthrough volume corresponds to the largest sample volume that can be processed without significant loss of analyte and for which recovery after elution for sample volumes less than the breakthrough volume will be 100% in the absence of irreversible sorbent interactions. It is the breakthrough volume that is most important in determining the suitability of an adsorbent medium for a particular isolation procedure. For practical purposes it will be preferred that the breakthrough volume of the first layer for the analyte is low. The breakthrough volume is in part determined by the nature of the reagent or solvent used. Suitable reagents or solvents used in the context of the present invention are discussed below.

In one embodiment of the devices and methods of the present invention, the nature of the solid support material of the second (detection) layer is similar to that of the first (clean-up) layer of the adsorbent medium or device. More particularly, in one embodiment both the first and the second layer comprise Sepharose. However, in the second (detection) layer of the device, the matrix comprises one or more analyte receptors bound to its surface which are not present in the first layer, whereby the surface of the second layer is typically saturated. Accordingly, the second layer used in the device and methods of the present invention, even if derivatized for the binding of the analyte-receptor thereto, is quenched and will bind only through the analyte-receptor on its surface.

According to a particular embodiment, the second layer of the device, which is used to specifically bind the analyte uses the immunoaffinity principle based on an antibody-analyte interaction. For instance, in case the analyte is a mycotoxin, the analyte-receptor according to this embodiment is an antibody specifically recognizing said mycotoxin. As an illustration, in Table 1 toxicants and other contaminants and matrices in which they occur are matched with their antibodies and exemplary companies they can be obtained from.

Antibodies useful in the context of the present invention include both monoclonal and polyclonal antibodies, capable of specifically recognizing immunologically active parts or specific epitopes of the analyte of interest. The term “specifically recognizing” implies that there is substantially no cross-reaction of the antibody with other components than the corresponding analyte. The antibodies according to the invention may be produced according to techniques which are known to those skilled in the art. Monoclonal antibodies may be prepared using conventional hybridoma technology as described by Kohler and Milstein (Kohler F. and Milstein C. Nature 256, 495; 1975). This classical method comprises producing hybridomas from, on the one hand, isolated splenic lymphocytes of an animal, particularly a mouse or a rat immunized against an analyte of the present invention or a fragment as defined above, and cells of a myeloma cell line on the other hand, and selecting a specific hybridoma for the ability to produce the monoclonal antibodies recognizing the analyte which has been initially used for the immunization of the animals.

Alternatively to preparing monoclonal antibody-secreting hybridomas, a monoclonal antibody directed against an analyte for use in the device and methods of the invention can be identified and isolated by screening a recombinant combinatorial immunoglobulin library (e. g., an antibody phage display library) with the analyte of interest.

Additionally recombinant antibodies, such as chimeric and humanized monoclonal antibodies, comprising both human and non-human portions, which can be made using standard recombinant DNA techniques, are within the scope of the invention.

According to an alternative embodiment the analyte-receptor is one of a receptor-ligand pair, i.e. a receptor (or ligand-binding fragment thereof) where the analyte is a ligand or a ligand (or receptor-binding fragment thereof) where the analyte is a receptor. According to yet another embodiment the analyte receptor is one of an enzyme-substrate pair, i.e. an enzyme (or substrate binding fragment thereof) where the analyte is a substrate of the enzyme or a substrate (or an enzyme-binding fragment thereof), where the analyte is an enzyme. Other analyte-specific molecules can be envisaged for use in the context of the present invention in the specific detection of analytes.

The binding of the analyte to the analyte-receptor in the detection layer can be determined by any one of a number of methods, described more in detail, in the context of the detection methods using the devices of the present invention below.

According to the present invention devices are provided which comprise a first set of one or more clean up layers and a second set of one or more detection layers. Preferably, the first set of layers is at least one centimeter thick while the second or subsequent layers are each preferably between 0.5 and 5 mm thick or less. In particular embodiments, at least one second layer is a membrane (e.g. polymeric membranes).

Specific embodiments of the devices and methods of the invention also provide or make use of devices wherein, in the second layer, at least a predetermined space/area of the second layer comprises a predetermined amount of the analyte molecule to be detected, the analyte molecule being labeled with an enzyme or a bioluminescent, chemiluminescent, phosphorescent or fluorescent molecule. Where the label is an enzyme, the enzyme is preferably similar to the enzyme which is used in the assay performed for the detection of the analyte in the second layer of the device of the invention. Such devices are useful in the detection of the presence or absence of an analyte in a sample, for instance because they provide for an internal standard, which may give a more quantitative estimation of the analyte present in the sample, and at the same time, may serve as a control for the reliability of the assay.

According to one embodiment, the methods and devices of the present invention comprise or involve the use of, as part of the second layer, an internal control layer. Accordingly, the second layer comprises (a) an anti-enzyme (internal control) layer and (b) an anti-analyte layer. The devices and methods thus may comprise the following layers (1) a solid phase clean-up and (2) immunological layer consisting of (a) an anti-enzyme layer and (b) an anti- analyte layer and their use in the detection of analytes.

Accordingly, one aspect of the present invention provides methods for detecting the presence or absence of one or more analytes in an interfering fraction-containing fluid or semi-fluid sample which involve the use of one or more “binder” molecules, i.e. molecules capable of specifically binding to the analyte-receptor. Such methods typically comprise the following steps:
(a) applying the sample in a flow-through motion onto an adsorbent medium comprising at least two sets of layers in which the first set of one or more layers is capable of actively adsorbing at least a part of the interfering fraction present in the sample, and the second set of one or more layers is capable of specifically retaining the one or more analytes of interest present in the sample; most particularly, at least part of the adsorbent medium of the first layer comprises a derivatized surface which is capable of actively adsorbing interferences, such as color, present in the sample,
(b) optionally washing the adsorbent medium in order to remove possible color interference of the second layer,
(c) optionally applying a predetermined amount of at least one binder molecule onto the adsorbent medium, each of the at least one binder molecules capable of interacting with a corresponding non-occupied analyte-receptor of the second layer, and optionally washing unbound binder molecule(s) from the adsorbent medium, and
(d) detecting the presence or absence of the one or more analyte(s) of interest.

In a specific embodiment of this aspect of the methods of the present invention, in step (a) of the above method, at least one of the second layers is capable of specifically retaining an analyte present in the sample by the presence of an antibody, specifically recognizing an analyte under investigation.

The detection step in the methods of the present invention may vary in nature, depending on how the analyte is to be detected. The analyte(s) bound onto the second layer of the devices of the invention can be detected in different ways. Some molecules can be detected directly, for instance aflatoxins emit fluorescent light under longwave UV.

An interesting embodiment of the invention, however, is a method in which the detection of the presence of the analyte of interest in the second layer can be done visually, for instance based on whether a color develops or not. Visual detection is essential for field testing and provides for quantitative or at least semi-quantitative results, for instance by detection or visual interpretation of different intensities of the color, for instance the blue color. This can be achieved by direct or indirect detection of the analyte bound to the analyte-receptor by making use of a molecule capable of generating a signal which is visually detectable. Direct detection methods typically involve detection of the analyte bound to the analyte-receptor. Indirect detection methods typically involve detection of the non-occupied analyte-receptor remaining in the second layer (by detection of labeled binder bound to the analyte-receptor after adding of the sample to the adsorbent medium). Alternatively, indirect detection can involve the detection of labeled binder not displaced from the analyte-receptor. Accordingly, the detection of the analyte either requires the addition of a labeled binder (capable of specifically binding to the analyte receptor) or of a labeled analyte-specific reagent (e.g. capable of specifically binding to the analyte as bound to the analyte-receptor). Examples of suitable labels include enzymes capable of reacting to produce a colored reaction product, such as horseradish peroxidase and alkaline phosphatase. Molecules capable of producing detectable light are also envisaged as labels, for instance molecules such as bioluminescence, chemiluminescence, phosphorescence and fluorescence, and particles such as carbon black, colored latex beads or gold particles. Where the label used is an enzyme, the methods of the present invention include the step of adding a suitable substrate. As detailed above, preferred substrates include those which generate a color which can be detected visually. Suitable substrate solutions include but are not limited to, for instance, a chromogen such as Color Burst©, p-Nitrophenyl Phosphate (pNPP), 5-Bromo-4-Chloro-3-Indolyl Phosphate/Nitro Blue Tetrazolium (BCIP/NBT), Fast Red/Naphthol AS-TR Phosphate, 2,2′-Azino-bis(3-Ethylbenzthiazoline-6-Sulfonic Acid) (ABTS), o-Phenylenediamine (OPD), 3,3′,5,5′-Tetramethylbenzinedine (TMB), 5-Aminosalicylic Acid (5AS), 3,3′-Diaminobenzidine Tetrahydrochloride (DAB), D(−)-Luciferin (for Bioluminescence), POD.

According to a particular embodiment of the methods of the invention, detecting the presence or absence of the analyte of interest in the second layer is done by the naked eye. Alternatively, a suitable detector means can be used, capable of electronically detecting the color developed and providing a more exact quantification. Such a quantification allows the calculation of the concentration of analyte in the test sample. Any detection method may be assisted by computer technology and detection methods can therefore be automated by various means. A suitable detector might be, for instance, a colorimeter.

It is further described in separate embodiments how the above more general method is used in combination with additional steps and reagents to obtain a variety of possible methods or assays.

For instance, alternatives of the method described above, are methods wherein step (b) is present, or step (c) is present, or wherein both step (b) and (c) are present.

According to a particular embodiment, in the methods of the invention, step (c) is replaced by a step (c′), wherein in step (c′) a predetermined amount of a binder molecule, labeled with an enzyme or a bioluminescent, chemiluminescent, phosphorescent or fluorescent molecule, is applied onto the adsorbent medium, the binder molecule capable of interacting with non-occupied analyte-receptor of the second layer. The binder is able to provide indirect detection of the absence or presence of the analyte of interest in the second layer. For this purpose, a suitable label is attached or conjugated to the binder, the label being detected and/or quantified.

In particular embodiments, the methods of the invention comprise a step wherein the sample under investigation is pre-treated by dissolving or extracting it with a specific solvent prior to applying the sample onto the adsorbent medium or the device of the present invention. In particular, the pretreatment step extracts, concentrates or dissolves the analyte present in the sample.

The nature of the solvent is selected such that, upon administration to the adsorbent medium, it is compatible with both the clean-up in the first layer and the detection in the second layer of the adsorbent medium in the methods and devices of the present invention. Optionally, the methods of the invention comprise a dilution step, whereby the solvent used for sample pre-treatment is diluted prior to contacting with the adsorbent medium. The choice of the adequate solvent for the detection and/or quantification of a given analyte using an adsorbent medium with a given first and second layer can be determined by the person skilled in the art, taking into account the following principles:
The adequate solvent can be a mixture of solvents.
The adequate solvent is preferably a good solvent for the analyte. A good solvent can be chosen by following the principle “like likes alike”, i.e. a good solvent will usually be a solvent having a polarity and/or chemical groups similar to the analyte. The choice of a good solvent for the analyte permits the efficient elution of the analyte. It is important that as little analyte as possible is retained in the first set of one or more clean-up layers.
The adequate solvent should, optionally upon dilution, also be compatible with the second set of layers. In particular, where the second set of layers comprises one or more analyte receptors that are proteinaceous, i.e. are susceptible to denaturation by some solvents, such solvents are preferably avoided or used at low concentration or in limited volumes. In particular, where the analyte receptor is an anti-body, aqueous solvents form preferably between 70 and 100% of the solvent and if organic solvents must be added (e.g. for solubility reasons), their proportion in the solvent mixture is preferably comprised between 0 and 30%, most preferably between 0 and 20%. Where the concentration of organic solvent is higher, the solvent is preferably diluted prior to administration to the adsorbent medium of the present invention. In a particular embodiment, a preferred organic solvent is methanol.
The adequate solvent should, as such or in diluted form also be compatible with the first set of one or more ‘clean-up’ layers, i.e. the solvent should preferably permit a good elution of the analyte and a good adsorption of the interfering fraction through the first set of layers.
If the interfering fractions are known, the solvent is preferably less good a solvent for the interfering fraction than for the analyte. This helps to reduce the amount of interfering fraction that will reach the second set of layers. This is of particular interest where the interfering fraction is or comprises a color pigment potentially causes colouring of the second set of layers.
The pH of the solvent should optimally be controlled. Indeed, the pH of the solvent can have a large impact on the elution of the analyte through the first clean up layer(s). This will particularly be the case if the first (detection) layer(s) comprise protonable/deprotonable layer(s) and/or if the analyte is itself protonable or deprotonable.

According to a particular embodiment, the breakthrough volume of the first layer(s) of the adsorbent medium is minimized. For field testing, elution of the analyte in the methods of the present invention is preferably ensured with a volume of less than 10 ml, more particularly less than 5 ml, most particularly less than 2 ml solvent.

In particular embodiments of the methods of the present invention, a washing step is included. More particularly, the adsorbent medium comprising the clean up and detection layers of the present invention is washed in order to remove all possible color interference of the second layer. In a specific embodiment, this washing is done using a conventional buffer, for example phosphate buffered saline, Tris buffered saline or water.

In those embodiments of the methods of the present invention which include the use of a binder, the binder can be such that it is detected directly (where the binder itself generates a detectable signal), or alternatively the binder is such that it can be detected after the addition of an additional component, e.g. a substrate (where the binder comprises an enzyme capable of converting a substrate into a molecule generating a detectable signal). In case a substrate is used to ensure detection of the binder, the different layers of the adsorbent medium are preferably washed prior to adding the substrate in order to remove all unbound binder from the second layer. This washing step can be done using a conventional buffer as described above.

As detailed above, a labeled binder can be used to ensure indirect detection of the analyte in the sample. Indeed, where the labeled binder is added after the sample has allowed to flow through the adsorbent medium, the labeled binder is captured by the non-occupied analyte-receptors of the second layer. Where the binder comprises an enzyme, after removal of the unbound binder and addition of the substrate, the substrate will react with the binder bound onto the non-occupied analyte-receptor of the second layer and generate a detectable signal. In the absence of any analyte in a sample under investigation, all the binder will be trapped in the second layer/detection zone yielding a high signal. The presence of analyte in the sample produces a decrease in signal proportionately as the amount of analyte in the sample increases. The intensity of the signal developed can be compared to that of known quantities of analyte applied to similar devices in the same manner and thus representing “reference” devices, or can be applied to a device including an internal standard as described above. For instance, the substrate can consist of a chromogen which is converted to a blue color by an enzyme conjugated to the binder. In this case, the interpretation of the result can be done visually and is based on whether a blue color develops or not. When the sample contains a particular amount of an analyte (or more), all analyte-receptors are occupied and accordingly no color develops. When the analyte concentration is lower than this critical concentration level, a blue color develops.

Accordingly, particular embodiments of the methods of the invention, comprise additional steps in between steps (c) or (c′) and (d) of the method as described above, such as, but not limited to:
optionally washing the adsorbent medium in order to remove all unbound binder from the second or detection layer, and
optionally applying a substrate solution onto the adsorbent medium, the substrate solution capable of reacting with the enzyme-labeled binder bound onto the non-occupied analyte-receptor of the second layer and capable of generating a detectable signal.

In a particular embodiment, the invention relates to any of the methods as described above wherein as one example of a binder molecule, a predetermined amount of the analyte molecule is applied onto the adsorbent medium to be detected, the analyte molecule labeled with an enzyme or a bioluminescent, chemiluminescent, phosphorescent or fluorescent molecule, and furthermore, the labeled analyte molecule capable of interacting with non-occupied analyte-receptor of the second layer. The addition of a labeled analyte molecule at that moment in the assay method, provides a means for detection of the absence or presence of the analyte of interest retained from the sample by the analyte-receptor in the second layer. Alternative embodiments of the binder molecule used in the context of the present invention include, but are not limited to a fragment or a variant of the analyte capable of binding to the analyte-receptor.

According to a particular embodiment of the methods and devices of the present invention, a pressure means is used to ensure that the sample is transported to the first and second layers of the adsorbent medium. Thus, according to these embodiments, the inlet means of the device of the invention are connectable to pressure means (9) capable of exerting pressure upon the sample to force the transport of the sample from inlet means (2) to outlet means (3). In FIG. 1, a pressure means (9) suitable for use in the invention is depicted. In this case, the pressure means consists of a syringe barrel (10), and the sample is applied onto the device (for instance, a tube) by means of a syringe plunger (11). Alternatively, in case the housing of the device is a syringe itself, the pressure means can consist of a syringe plunger which fits into said syringe. An alternative embodiment of the devices of the present invention comprises a pressure means which is a vacuum means for aspiring the sample from the inlet means to the outlet means, as illustrated in FIG. 6.

This invention thus employs frontal elution or elution chromatography. As pressure or vacuum is continuously applied by e.g. the plunger, the mobile phase carries the dissolved analyte towards the outlet means (3), for instance the end of the tube in FIG. 1 or the opposing end of the tube in FIG. 6. As a result the analyte(s) of interest is/are quickly loaded onto the second set of one or more adsorbent layers of the device where it/they will selectively bind to the analyte receptor(s). In this chromatographic elution system the breakthrough volume is significantly reduced.

The device and method of the present invention have several advantages. These advantages mainly result from the fact that the present invention permits a simultaneous clean-up and detection of one or more analytes in a sample under investigation. In addition, in particular embodiments, interpretation of the results can be done visually. Furthermore, the device of the current invention is easy to fabricate using readily available, relatively inexpensive materials. Moreover, the test method which employs this device is rapid and easily performed. The reagents and equipment needed for said method are portable and stable at ambient conditions and safe to use. Yet another important advantage is that the device and method are particularly useful for field testing and screening of samples for the presence of analytes, without the need for extensive training or expensive laboratory equipment.

In summary, the screening method and device of the invention provides fast, simple, cost-effective and reliable information when operated under field conditions

The invention can be applied as a general detection method for a large variety of target analytes. For instance, the devices and methods can be used for the detection of toxins or mycotoxins in food and feed, for pesticides in water, hormones and antibiotics in milk or body fluids. The devices and methods of the invention will prove useful as a regulatory tool to monitor mycotoxin contamination in agricultural commodities, prepared foods and mixed feeds at buying locations, field installations, processing lines, grain elevators, feed lots and the like. It can facilitate the rapid differential diagnosis of mycotoxicoses in animals (by testing body fluids or tissue extracts, particularly those of the liver and kidney) and perform presumptive field analyses for mycotoxins.

Furthermore, the invention provides easy-to-use devices and methods in doctors offices, at clinics or at home for testing the presence of hormones or therapeutic drugs in body fluids. Additionally, clinics, emergency medical technicians and policemen require an affordable and easy to use device for quickly testing for the presence of drugs of abuse in body fluids outside of a hospital setting.

According to a general embodiment the invention thus relates to any of the methods described herein in which the analyte in said sample under investigation is a member selected from the group consisting of toxins, mycotoxins, pesticides, drugs, antibiotics, hormones, and their respective conjugates, metabolites and derivatives.

The invention is further illustrated in the enclosed example describing the use of the method and device for screening for the mycotoxin ochratoxin A (OA) in roasted coffee. However, it should be appreciated by those skilled in the art that this example is merely illustrative and a great variety of embodiments are possible which employ various combinations of adsorbents and antibodies depending on the various analytes for which analysis is desired.

The housing of the device in this particular example is a tube containing two superposed adsorbent layers. The roasted coffee samples are first extracted with an appropriate organic solvent, and after dilution of the solvent, applied onto the tube. The adsorbent material of the first layer will trap all possible interferences and clean-up the sample. Next, a washing solution is applied onto said tube to remove all color of the second layer. The second layer of the device uses an immunoaffinity principle based on an antibody-analyte interaction system. An antibody specifically recognizing OA is covalently bound onto the adsorbent material of the second layer. Ochratoxin present in the sample under investigation will be retained onto said second layer. Next, an amount of a labeled OA solution is applied onto the tube and will bind non-occupied antibody sites of the second layer. The tube is again washed in order to remove unbound ochratoxin. Detection can be done by the naked eye, after applying a substrate solution onto the tube capable of reacting with labeled ochratoxin bound onto the second layer of the tube. When no analyte was present in the sample under investigation, the second layer of the tube will color strongly. No or less color develops when the analyte concentration increases in the sample.

Notwithstanding the inventive concept of applying the clean-up method and a detection method in a single device and assay, the cleaning up method has been optimized by the present inventors and can be used separately for instance for removing an interfering fraction from a fluid or semi-fluid sample, prior to the analysis of said sample in HPLC or in a conventional or flow-through enzyme immunoassay.

In general solid phase extraction (SPE) procedures, a sample with analyte is applied over the solid phase, the targeted analyte binds to the solid phase while the rest of the sample passes through the solid phase, the solid phase is washed with buffer to remove interfering matrices, the analyte is then eluted with an appropriate solvent, the eluate is evaporated to dryness and re-dissolved in a smaller volume to pre-concentrate it for analysis.

The clean-up extraction method of the present invention is only a two step procedure wherein the sample is processed in a directly opposite way with the sample extraction solution used directly as the analyte eluate by providing a conducive solvent environment for the analyte. The sample is brought to the analytical immunoaffinity layer directly at a low alcohol concentration thus does not affect the immunological reactions of the second layer. This is thus only a two step method in as far as the solid phase clean-up procedure is concerned compared to the mainstream SPE principles.

Therefore, according to another embodiment, the invention relates to a solid phase cleaning up method for removing an interfering fraction from a fluid or semi-fluid sample, said method comprising applying the sample in a flow-through motion onto an adsorbent medium which is capable of actively adsorbing at least a part of the interfering fraction of said sample, characterized in that said adsorbent medium comprises a solid support material selected from the group consisting of silica derivatives and wherein the surface of said solid support material is derivatized to produce a bonded matrix, for instance a bonded matrix wherein the functional chemical groups displayed at the surface of the solid support medium are chosen from octadecyl, octyl, ethyl, cyclohexyl, phenyl, aminopropyl, cyanopropyl, diol, n-propyl-ethylene-diamine, diethylaminopropyl, benzenesulfonylpropyl, sulfonylpropyl, carboxymethyl and trimethylaminopropyl.

The invention also relates to a device operable in the above described method, for instance a device wherein said absorbent medium is a bonded silica solid phase e.g., aminopropyl solid phase.

In an interesting embodiment, said cleaning up method and/or said device are used with a sample wherein the presence or absence of ochratoxin A needs to be detected.

Aminopropyl bonded silica is prepared by the reaction of the silanols with halo- or alkoxylyl derivatives, resulting in the covalent bonding of a wide range of functional groups. The high surface coverage that can be achieved during the bonding process means that the adsorptive characteristics of the bonded silica sorbent are largely a function of the characteristics of the phase covalently bonded to the silica surface:
—Si—CH₂—CH₂—CH₂—NH₂

Aminopropyl is a polar solid-phase and the polarity is due to a concentration of negative charge on one end of the molecule and a concentration of positive charge on the other end. This is brought about by having the negative electrons within the atoms of the molecules shift towards those molecules which are most capable of attracting them. This shift produces a molecular dipole. Other polar molecules will be attracted since each of them, in turn, have a positive and negative end. In aminopropyl the polar characteristic is brought about by the amino-group. Amines have at least one sp3 hybridized nitrogen bond to bond to as few as one hydrogen group. The nitrogen atom of primary amines has a lone pair of electrons that often in the presence of a more acidic substance is capable of donating the lone pair in forming a fourth bond making the nitrogen atom electron deficient giving it a net positive charge.
CH₃—NH₂+H⁺→CH₃—NH₃⁺

This sets the aminopropyl solid phase ready to receive an electron from a polar compound. It 25 is for this reason that for instance in particular applications, such as for detecting OA, the extraction and hence the elution solution should contain a high concentration of methanol in order to directly elute the bulk of the OA in the extract at levels that are detectable by the enzyme immunoassay. At lower methanol concentrations lower amounts of methanol interact with both OA and aminopropyl solid phase to effectively dissolve it. At higher methanol concentrations it is envisaged that OA and the aminopropyl solid phase will be completely associated with methanol molecules to effect a direct elution as the interaction of OA with the solid phase will be somewhat impeded by the methanol. Methanol has a dipole moment of 1.6, polarity of 232.3 kJ.mol⁻¹ and a nucleophilic donor strength of 107.5 kJ.mol⁻¹. These are apparently higher than those for the amino-nitrogen. The association of OA with methanol in the sample is, therefore, expected to receive the minimum disruption as it passes through the column.

Although aminopropyl has a short carbon chain it is capable of limited non-polar interactions. Also according to the invention, the cleaning up method described above is used for actively removing an interfering fraction from said sample in a method for detecting the presence or absence of an analyte in a fluid or semi-fluid sample, for instance prior to the application of said sample in a flow-through enzyme immunoassay or in an HPLC analysis.

The immunoaffinity principle is a basic principle for instance used in immunoaffinity columns (IAC). These columns contain a bed of a solid support material to which anti-analyte antibodies are covalently bonded. A sample containing the analyte is applied onto the column and the antibodies specifically bind the analyte after which all unbound materials are washed off and the analyte is finally eluted separately. The eluate is taken for analysis by either ELISA, RIA, HPLC, LC-MS or GC-MS. Enzyme-linked immunosorbent assays (ELISA) are also based on antibody-analyte interactions and can be used for both qualitative and quantitative analyses. These are formatted as microtitre plates, the assay and results of which are carried out and interpreted in a laboratory environment using a microtitre plate reader. Other antibody-antigen systems include immunochromatographic systems comprising of a membrane along which the analyte diffuses until it reaches a site on the membrane where the anibody is bound. In another ELISA format achronymed enzyme-linked immunofiltration assay (ELIFA) or flow-through immunoassay the sample is applied directly onto a membrane where the antibody is spotted while an absorbent material draws the samples through the membrane bringing the analyte to the antibody sites. These assays can be carried out in both the lab and field and results interpreted visually. Radio immunoassays (RIA) also utilize the antibody-antigen reaction system, and are highly sensitive. However, their detection system utilizes radioactive decay which may result in handling problems. RIA applications are mostly in cell biology, for example, signal transduction and cytoplasm-based assays for the analyte detection and quantification.

However, these immunological assays of the prior art clearly differ from the methods of the present invention. In these assays an extraction and clean-up step usually precedes and is performed separately from the immunological assay itself. On the contrary, in the present invention the solid phase clean-up step and immunoassaying of the sample are carried out simultaneously in a single assay and in one single device containing the two different types of layers as described above. The analyte(s) is/are largely prevented from binding to the bonded stationary phase of the first layer and therefore pass(es) directly onto the second layer, while interferences stay bound on the solid phase part of the first layer.

The invention, now being generally described, will be more readily understood by reference to the following examples, which are included merely for purposes of illustration of certain aspects and embodiments of the present invention and are not intended to limit the invention.

### EXAMPLES

### Example 1

### Device and Method for Detection of an Analyte in a Interfering Fraction Containing Sample

Since different types of gels offer various options for immunological detection, they make it possible to prepare a range of detection layers for use in the devices. These gels include CNBr-activated Sepharose 4B, Activated CH Sepharose 4B, NHS-activated Sepharose and EAH Sepharose 4B (for aminopropyl group attachment), ECH Sepharose 4B (for —COOH group attachment), while Epoxy-activated Sepharose 6B couples through hydroxyl, amino or thiol group, all these are for instance available from Pharmacia (Sweden).

A ligand (analyte receptor) solution with a specified concentration is coupled with a specified amount of gel by a method described by the manufacturer. The choice of analyte receptors, for instance antibodies, depends on the analyte to be detected and are commercially available from several suppliers.

Briefly, the gel is swelled by washing with 1 mM HCl (200 ml/g) over a sintered glass filter, the ligand is dissolved in an appropriate buffer, for instance 0.1 M NaHCO₃ (pH 8.3) buffer containing 0.5 M NaCl (5 mg ligand ml⁻¹ gel) and subsequently coupled with gel by shaking end-over-end for 2 hrs at RT or overnight at 4° C. The remaining active sites are blocked, for instance with either 0.2 M glycine (pH 8.0) or 1 M Ethanolamine for 16 hrs at 4° C. or 2 hrs at RT. Excess adsorbed ligand is washed away for instance with coupling buffer followed by 0.1 M acetate (pH 4) buffer containing 0.5 M NaCl followed by coupling buffer. The blocking agent is washed away using coupling buffer. The coupled gel/buffer ratio is for instance 1/3.

A separate amount of gel is swelled and blocked as described above. The coupled gel (for instance 1 ml in 3 ml buffer) is mixed with 4 times this volume of blocked gel. The mixture is brought to an equilibrium by shaking end-to-end for 3 minutes. For instance 150 μl of this mixture is pipetted into an empty column with the endcap and first grid in place (FIG. 1). Empty columns with grid can be obtained, for instance, from Varian, (Harbor City, USA). The second grid is introduced to compress the gel suspension to a final thickness of approximately 2 mm (FIG. 1). The solid phase material (for instance 200 mg) (clean-up layer) is introduced after the column is filled with buffer, for instance NaHCO₃ (FIG. 1). Various types of solid phases, for instance bonded Solid phases (anionic, ionic, polar and non-polar) are obtainable from Varian (Harbor City, USA) and J. T. Baker (Belgium). The third grid is introduced to superimpose over the solid phase material adequately compressed (FIG. 1). At this stage the column is ready for use.

A sample is collected and dissolved/extracted with a specific amount of solvent which in turn is compatible with the immunoaffinity section of the column (either as such or further to dilution). The sample is applied on the column through a syringe by means of a syringe plunger (FIG. 1) (Syringes are for instance from Becton Dickinson, (Temse, Belgium)).

First the sample encounters a specified amount of solid phase onto which sample matrices are adsorbed while the analyte is favorably dissolved in the solvent. The solvent flows through the solid phase part of the column (first clean-up layer) carrying the analyte to the immunoaffinity section (second detection layer). The compatibility of the solvent with the immuno-reactive section part of the column ensures that the antibodies are not affected and the analyte is bound. Any discoloration and matrices are washed off with a specified amount of washing buffer. A specified amount of enzyme-analyte concentration is applied onto the column. Any unbound enzyme conjugate is washed off with washing buffer. Then a volume of chromogen substrate is applied and a color develops on the immunoaffinity section of the column for samples pre-defined as negative and no color develops for positive samples. Chromogen substrates can be obtained for instance from Sigma (USA), Calbiochem (San Diego, USA), Pierce (Belgium), or other suppliers.

### Example 2

### Development of a Solid Phase Immunoaffinity Column-based Enzyme Immunoassay for the Detection of Ochratoxin A in Roasted Coffee

A column and a method for a simultaneous clean-up and analysis/detection of OA was designed.

The main principle is to use the stationary bonded phase to adsorb and trap matrix interferences while the analyte remains dissolved in the mobile phase and is subsequently adsorbed by the immunoaffinity section. Therefore, to effectively increase the sensitivity of the assay the sample is diluted with a solvent which dissolves the analyte. Thus the diluent creates an environment most favorable to the analyte. This decreases the solid/mobile phase partition coefficient in favor of the mobile phase. The molecule is then directly eluted as the sample passes through the immunoaffinity section of the column. The matrix interferences are retained on the solid phase by the specific modes of interactions provided for by the chemical environment.

This method employs frontal elution or elution chromatography. As pressure is continuously applied on the plunger the mobile phase carries the dissolved analyte towards the outlet end of the column. Thus, the analyte of interest (OA) is quickly loaded onto the immunoaffinity section of the column where it is selectively bound and any remaining interfering substances are washed off. In this chromatographic elution system the breakthrough volume is significantly reduced. It is the breakthrough volume that is most important in determining the suitability of a sorbent for a particular isolation procedure. As demonstrated by the partition coefficient (Table 5) and the elimination of interfering peaks the aminopropyl solid phase material was reliably adopted for use in this column.

The second part of the column to which the analyte binds uses an immunoaffinity principle based on an antibody-analyte interaction system.

In the present method the solid phase clean-up and the immunoassaying of the sample are carried out simultaneously in the same column.

A column as generally described in Example 1 was prepared and the method was optimized, for instance, to more specific requirements for detecting OA in a sample of roasted coffee.

Anti-Ochratoxin A antibodies and Horse Radish Peroxidase (HRP)-OA conjugate were obtained from the Institute of Animal Sciences, Agricultural Biotechnology, (Gödöllö, Hungary). The anti-OA was coupled with the CNBr-activated gel (Pharmacia Biotech, Sweden) (for preparing the detection layer) and diluted with blocked gel as described in Example 1. Aminopropyl (solid phase material for preparing the clean up layer) was obtained from J. T. Baker (Belgium) while ColorBurst® Blue was obtained from ALerCHECK Inc. (USA). Ground roasted coffee (5 mg) were extracted with 15 ml 50% Methanol/3% aqueous NaHCO₃ by shaking for 15 minutes. The sample was filtered with filter paper and 8 ml of extract were diluted to 20 ml with 3% aqueous NaHCO₃ to reduce the MeOH to 20%. The dilution was applied over the column at a rate of 1 drop per second. Subsequently, the column was washed with 10 ml of 3% aqueous NaHCO₃ and 100 μl of HRP-OA (1:200) in assay buffer (0.1% Casein PBS pH 7.4) was added. The column was washed once more with 10 ml of H₂O and a 50 μl volume of ColorBurst® Blue was drawn into the immunoaffinity section of the column by withdrawing the syringe plunger such as to create a backward flow or sucking action through the tip of the column. The chromogen substrate fills the immuno-reactive part of the column.

The method was further optimized as follows.

The effects of volume of sample extract were investigated in an assay by comparing 10 ml of PBS (pH 7.4) to 2, 4, 6 and 8 ml of roasted coffee extract.

The different volumes were applied onto the columns by a syringe (obtained from Becton Dickinson, Temse, Belgium), and the assay was performed essentially as described above. The syringe provided the pressure means as outlined in FIG. 1.

An intense blue color was observed for the assay in which 10 ml of PBS (pH 7.4) was applied and for the assay in which 2 ml of sample extract was applied. There was less color development in the assays for which 4, 6, and 8 ml were applied.

To carry out the assay with a total elimination of cross-reactivities 2 ml of sample extract was chosen as the most definitive assay volume to be assayed.

In order to yield more OA from the column a higher concentration of methanol was used so as to effect a higher frontal elution of OA onto the immunoaffinity section of the column. However, increasing the concentration of methanol in the sample extract to be assayed may affect the immunoaffinity section of the column hence the performance and success of the assay. To optimize the concentration of methanol in an assayed sample extract spiked with OA, the effect of different concentrations of methanol on the performance of the assay were evaluated. The concentrations investigated were 10, 15, 20 and 25% methanol.

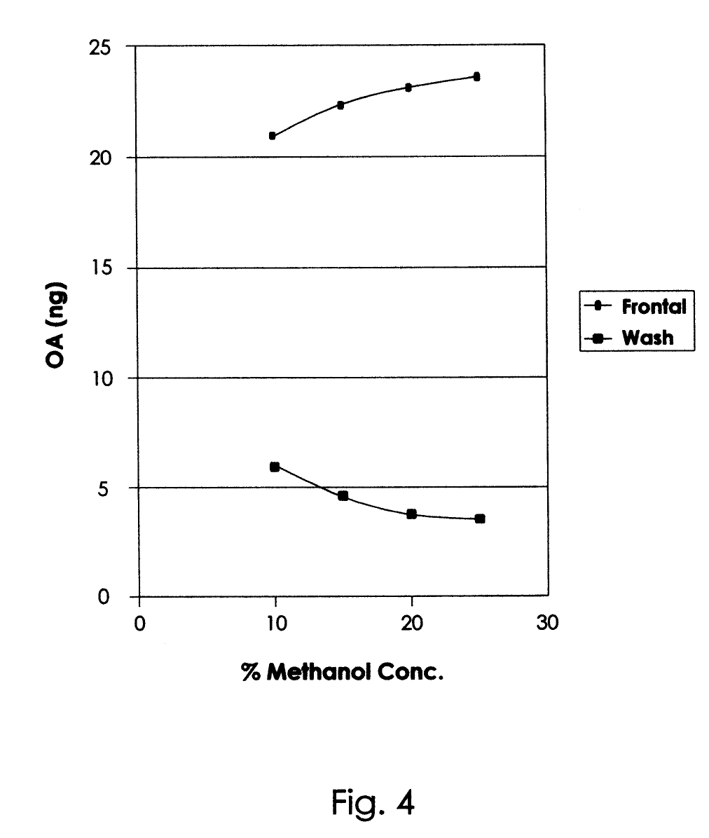

The retention capability of the aminopropyl column was greater at lower methanol concentration and tended to decrease as the methanol concentration increased from 10 to 20% (FIG. 4). The eluotropic ability of the sample extract increased with decreasing dilution factors. Though the eluotropic ability of the extract increased with increasing methanol concentration a methanol concentration of 20% was finally adopted so as to avert the negative effects of higher methanol concentrations on the immunoaffinity section of the column.

The recoveries of residual OA from the column with increasing methanol, as shown by the “wash graph” in FIG. 4, increased at a decreasing rate. Previous experience with enzyme immunoassays employing higher methanol concentrations showed no effect on the assay itself (Sibanda et al. 2000, J. Agricultural and Food Chemistry, 48: 5864-5867).

Therefore, in this case a conservative 20% methanol was chosen for use in the column-based enzyme immunoassay. The choice of a 20% methanol concentration was to ensure an efficient eluotropic effect on OA over the aminopropyl column. This thus carries a considerable quantity of OA to the immunoaffinity section of the column hence an increased assay sensitivity.

Furthermore, it was found that were no false positives recorded during the repeatability studies of the assay during 5 days (Table 6). The assay repeatability is high showing definite reliability of the assay and its applicability to routine screening of roasted coffee samples for OA.

### Example 3

### Optimization of Solid Phase Clean-up Method

The effect of pH and methanol on the direct elution of OA over the aminopropyl solid phase material was investigated by using three different methanol concentrations (40, 50 and 60%) for extraction and adjusting the sample extracts to different pH levels. The clean-up procedure used was as follows with different pH value. The extracts (mean pH 5.6) were adjusted to pHs 4.0, 4.5, 5.0, 5.5, 6.0, 6.5, 7.0, 7.5 and 8.0 using hydrochloric acid (HCl) and sodium hydroxide (NaOH). The sample, 3.5 ml, with the adjusted pH was passed over the aminopropyl solid phase material (200 mg) at a rate of 1 drop per second. The eluate was diluted to 60 ml with PBS (pH 7.4) and brought over an Ochratest™ IAC. OA was eluted with 4 ml of methanol. The eluate was evaporated at 40° C. under a stream of nitrogen gas and the residue was redissolved in 150 μl of methanol.

After processing, the samples were analyzed with the HPLC method.

There was no link between pH and OA recoveries over the pH ranges investigated. At 40% methanol/3% aqueous NaHCO₃ recoveries were almost nihl. However at 50% and 60% methanol recoveries increased significantly. At 50% methanol better recoveries were obtained over the pH ranges 5.0-6.0 which are relevant to a freshly extracted sample.

As it was evident, pH did not have a significant influence over the chromatographic elution of OA from aminopropyl columns. There was, therefore, no need to alter the pH of the extracts. For the detection of OA, it was however important that 50% methanol concentration be adopted as the working concentration for clean-up. The main reason being the need to dilute less to circumvent the reduction of assay sensitivities.

Further, the adsorptive and clean-up characteristics of the aminopropyl solid phase were optimized by means of establishing the most conducive chemical environment in which the interfering compounds are effectively bound. This was done by investigating the effect of increasing the NaHCO₃ concentration from 0% to 8% in the extraction solution. The extracts were cleaned-up and the eluates analyzed by HPLC to determine the dispersion of OA between the mobile and the stationary phase. This was achieved by collecting the mobile phase fraction (frontal fraction) and the stationary phase fraction (wash fraction) separately. The extraction and clean-up methods was as follows. The sample was extracted with 50 ml of methanol/0, 1.5, 3, 4, 6, and 8% aqueous NaHCO₃ (1/1, vol/vol). After filtration, 4 ml was extracted over 200 mg of an aminopropyl column at a rate of 1 drop per second into a test tube (frontal fraction). The column was washed with 2 ml of methanol/3% aqueous NaHCO₃ (1/1, vol/vol) and lastly with 1 ml absolute methanol and both washings were eluted into the same test tube. The eluate, 7 ml, was diluted to 100 ml with PBS (pH 7.4) and extracted over an Ochratest™ IAC and prepared for HPLC analysis.

Increasing the concentration of NaHCO₃ had an effect of increasing the adsorptive efficiency of the aminopropyl solid phase material. Although peaks were detected from 0% to 4% aqueous NaHCO₃ there were no peaks at 6% and 8% aqueous NaHCO₃. These results are shown in Table 3.

Since the peak disappeared completely when moving from 4% to 6% aqueous NaHCO₃, 5% aqueous NaHCO₃ was chosen as the optimum concentration and no matrix interference peak appeared afterwards.

### Example 4

### Optimization of Solid Phase Clean-up Prior to HPLC Analysis of Ochratoxin A in Roasted Coffee

The introduction of more sensitive High Performance Liquid Chromatography (HPLC) methods permitted the detection of trace levels of OA in roasted coffee (Terada et al. J. Assoc. Anal. Chem., 69 (1986) 960). However, the analysis of OA in coffee is still hampered by acidic substances extracted together with OA (Pittet et al. J. Agric. Food Chem. 44 (1996) 3564). The HPLC method was recently improved by the introduction of the use of immunoaffinity columns (IACs) for the clean-up of coffee products (Nakajima et al. Food Agric. Immunol. 2 (1990) 189). In a 1996 study a method was reported in which IACs were used directly after sample extraction without a clean-up step (Pittet et al. J. Agric. Food Chem. 44 (1996) 3564). Due to extensive interferences by the coffee matrix it was necessary to increase the retention time of OA to nearly 14 minutes. Later in 1997 the use of a Sep Pak Silica column for solid phase clean-up of the extract was reported and the resultant chromatograms showed a well resolved OA peak and a stable baseline (Patel et al. Food Addit. Contam. 14 (1997) 217). However, this clean-up method employed extensive washing steps using chloroform, chloroform-methanol, toluene-acetic acid and acetonitrile. In this example we describe a new clean-up method employing aminopropyl (NH₂) as the solid phase material. The method employs only three steps resulting in a sample extract which can be analyzed directly by an immunological method or further extracted by IAC for HPLC analysis.

The newly developed extraction method was used as a clean-up step prior to sample preparation for HPLC analysis. An interfering compound with a similar retention time as OA was adsorbed by the aminopropyl (NH₂) material at ≦5% NaHCO₃.

The main objective was to assess the extraction method and compare recoveries to standards representing the actual and expected quantities.

In the experimental set up, 20 g samples spiked with 0, 2.5, 5, 10, 20 and 40 ng OA.g⁻¹ were used. Samples were extracted with 50 ml of methanol/5% aqueous NaHCO₃ (1/1, vol/vol). The column was washed with 2 ml of methanol/5% aqueous NaHCO₃ (1/1, vol/vol) into the same flask and finally with 1 ml absolute methanol.

The HPLC method used was an adaptation of that described by Pittet et al. 44 (1996) 3564) The sample (50 μl) was injected manually by means of a Rheodyne manual injector (Waters, Milford, Mass., U.S.A). The HPLC system consisted of a Waters™ 600 Controller and a Waters 610 Fluid Unit (Waters, Milford, Mass., U.S.A.). The flow rate was 1 ml per min over a Supelco Discovery™ C18 (25 cm×4.6 mm, 5 μm) reversed-phase column (Supelco, Bellafonte, U.S.A.) at ambient temperature. The mobile phase used was acetonitrile/water/acetic acid (99/99/2). OA detection was achieved by means of a Waters 474 scanning fluorescence detector (Waters, Milford, Mass., U.S.A.) set at 333 nm excitation and 460 nm emission wavelengths.

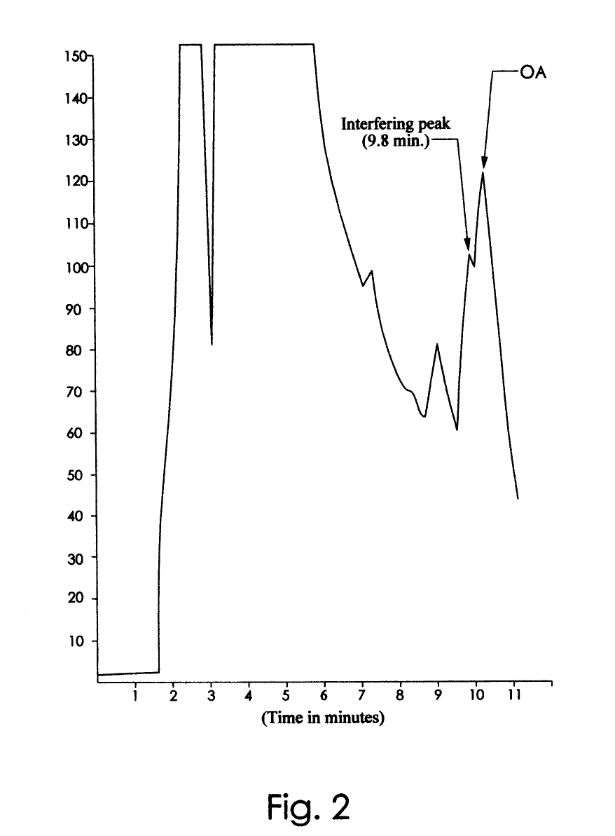

The HPLC conditions allowed retention of OA only up to 10 minutes. However, roasted coffee matrix interferences covered the chromatogram from ca. 1.5 minutes to over 15 minutes. There was a matrix peak with an identical retention time as that of OA in a blank roasted coffee sample after the IAC sample clean-up. This, therefore, masks the OA peak at ca. 10 minutes, which appears as a shoulder on matrix peaks (FIG. 2). This, therefore, illustrates the inadequacy of using IACs in isolation as a clean-up method highlighting the need to add an SPE step prior to the IAC clean-up step.

Various solid phases [trimethylaminopropyl (SAX), n-propyl-ethylene-diamine (PSA), aminopropyl, octadecyl (18), Diol (20H) and cyanopropyl (CN] were investigated for their ability to adsorb the matrix interferences and particularly the brown coffee color and the compound interfering with the OA peak 5 (Table 2). Aminopropyl was selected for its chromatographic elution of OA and adsorption of the interfering compound. Different concentrations of NaHCO₃ were investigated and there was an observed decrease in peak area of the interfering compound with increasing NaHCO₃ concentrations (Table 3). From these results 5% aqueous NaHCO₃ was chosen as the optimum salt concentration in the extraction solution for the effective adsorption of the interfering compound on the aminopropyl solid phase material.

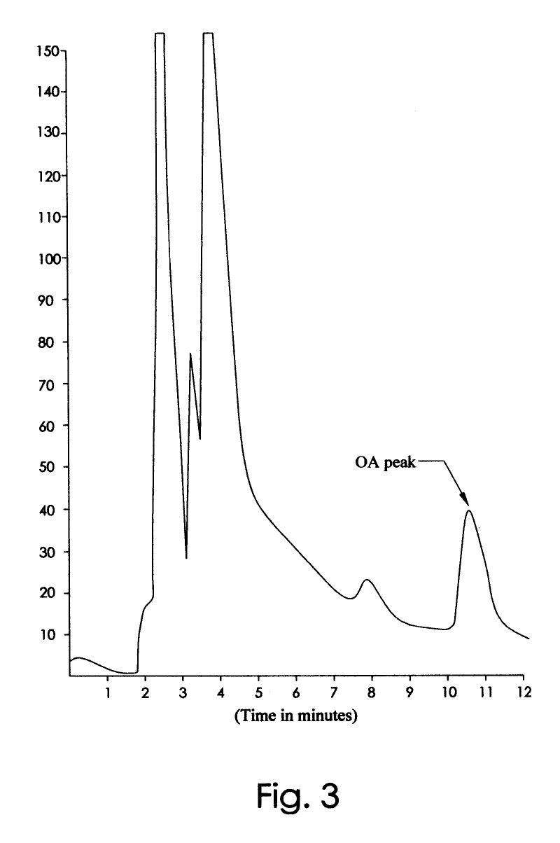

The chromatogram showed extensive elimination of matrix interferences resulting in a well resolved OA peak (FIG. 3). Method recoveries ranged from 72-84% in spiked samples (n=3 replicated twice) (Table 4). Regression (r²) of peak area on concentration for both standards and spiked samples were identical and these were 0.981 and 0.984, respectively. The recovery of OA from the aminopropyl column followed was confirmed by derivatization to its methyl ester confirmed whereas the interfering compound peak disappeared after derivatization. The method was successfully applied to 9 commercial roasted coffee samples. The main advantages here are that the quantification of OA can be done within half the usual time it takes to analyze roasted coffee samples. The column is not overloaded and the chromatogram shows that the baseline is established first before the OA peak appears.

Recoveries were considered high enough (Table 4) for the method to be used in the analysis and confirmation of roasted coffee samples. The clean-up method employing the aminopropyl solid phase material offers an efficient system for eliminating complex matrix interferences, therefore, there is no need for an extra confirmation step as required by other published methods for the analysis of similar matrices (Tsubouchi et al. J. of Agric. Food Chem. (1998) 36: 540; Studer-Rohr et al. J. of Food and Chemic. Toxic. (1995) 33:341; Pittet et al. 1996 J. Agric. Food Chem. 44 (1996) 3564). There is also no need to switch to different mobile phases when analyzing green and roasted coffee samples. The aminopropyl column clean-up method is also compatible with the rapid field enzyme immunoassay format. No pre-conditioning of the aminopropyl column is required. The ability of the clean-up method to chromatographically elute the toxin while adsorbing matrix interferences does not require an additional methanol step. This, therefore, has an advantage of requiring lower dilution factors and method sensitivity is not affected.

### Example 5

### Device and Method for Detection of Two Analytes in a Interfering Fraction Containing Sample

A ligand (analyte receptor) solution with a specified concentration is coupled with a specified amount of gel by a method described by the manufacturer. The choice of analyte receptors, for instance antibodies, depends on the analyte to be detected and are commercially available from several suppliers.

Briefly, the gel is swelled by washing with 1 mM HCl (200 ml/g) over a sintered glass filter, the ligand is dissolved in an appropriate buffer, for instance 0.1 M NaHCO₃ (pH 8.3) buffer containing 0.5 M NaCl (5 mg ligand ml⁻¹ gel) and subsequently coupled with gel by shaking end-over-end for 2 hrs at RT or overnight at 4° C. The remaining active sites are blocked, for instance with either 0.2 M glycine (pH 8.0) or 1 M Ethanolamine for 16 hrs at 4° C. or 2 hrs at RT. Excess adsorbed ligand is washed away for instance with coupling buffer followed by 0.1 M acetate (pH 4) buffer containing 0.5 M NaCl followed by coupling buffer. The blocking agent is washed away using coupling buffer. The coupled gel/buffer ratio is for instance 1/3. A separate amount of gel is swelled and blocked as described above. The coupled gel (for instance 1 ml in 3 ml buffer) is mixed with 4 times this volume of blocked gel. The mixture is brought to an equilibrium by shaking end-to-end for 3 minutes. For instance 150 μl of this mixture is pipetted into an empty column with the endcap and first grid in place (FIG. 1). Empty columns with grid can be obtained, for instance, from Varian, (Harbor City, USA). The second grid is introduced to compress the gel suspension to a final thickness of approximately 2 mm (FIG. 5). A second gel suspension prepared as described above but coupled to another ligand for a second analyte to be detected is introduced above the second grid. A third grid is introduced to compress the gel suspension to a final thickness of c.a. 2 mm (FIG. 5). The solid phase material (for instance 200 mg) (clean-up layer) is introduced after the column is filled with buffer, for instance NaHCO₃ (FIG. 5). Various types of solid phases, for instance bonded Solid phases (anionic, ionic, polar and non-polar) are obtainable from Varian (Harbor City, USA) and J. T. Baker (Belgium). The fourth grid is introduced to superimpose over the solid phase material adequately compressed (FIG. 5). At this stage the column is ready for use. A sample is collected and dissolved/extracted with a specific amount of solvent which in turn is compatible with the immunoaffinity section of the column (either as such or further to dilution). The sample is applied on the column through a syringe by means of a syringe plunger (FIG. 1) (Syringes are for instance from Becton Dickinson, (Temse, Belgium)).

First the sample encounters a specified amount of solid phase onto which sample matrices are adsorbed while the two analytes are favorably dissolved in the solvent. The solvent flows through the solid phase part of the column (first clean-up layer) carrying both analytes to the immunoaffinity section (second and third detection layers). The compatibility of the solvent with the immuno-reactive section part of the column ensures that the antibodies are not affected and the analytes are bound. Any discoloration and matrices are washed off with a specified amount of washing buffer. A specified amount of enzyme-(first)analyte concentration is applied onto the column. Any unbound enzyme conjugate is washed off with washing buffer. A specified amount of enzyme-(second)analyte concentration is applied onto the column. Any unbound enzyme conjugate is washed off with washing buffer. Then a volume of chromogen substrate is applied and a color develops on the second layer of the column for samples pre-defined as not containing the first analyte and no color develops for positive samples. Similarly, a color develops on the third layer of the column for samples pre-defined as not containing the second analyte and no color develops for positive samples.

### Example 6

### Development of a Solid Phase Immunoaffinity Column-based Enzyme Immunoassay for the Detection of Ochratoxin A (OA) and Aflatoxin B1 (AfB1) in Spices.

A column and a method for a simultaneous clean-up and analysis/detection of OA and AfB1 was designed.

The main principle is to use the stationary bonded phase to adsorb and trap matrix interferences while both analytes remain dissolved in the mobile phase and are subsequently adsorbed by the immunoaffinity section. Therefore, to effectively increase the sensitivity of the assay the sample is diluted with a solvent which dissolves both analytes. Thus the diluent creates an environment most favorable to the analytes. This decreases the solid/mobile phase partition coefficient in favor of the mobile phase. The analytes are then directly eluted as the sample passes through the immunoaffinity section of the column. The matrix interferences are retained on the solid phase by the specific modes of interactions provided for by the chemical environment.

For this method, a device according to FIG. 6 was used. As the plunger (11) is progressively pulled from the syringe barrel (10), a solution containing the dissolved analytes as well as interfering substances is drawn into the tube (1) through the inlet mean (2). After that the solution passed through all layers (4, 5 and 14), pressure was applied on the plunger (11) to flow the solution through the column again toward the inlet means (2) which acted here therefore also as an outlet means. This way, the interferences remained on the clean-up layer (4) and the solution was twice in contact with the detection layers (5 and 14). The analytes (OA and AfB1) were therefore quickly loaded onto the immunoaffinity section (i.e. the second set of layers) of the column where they were selectively bound. To remove interfering substances form the second set of layers, a washing step was performed in the direction outlet means (3) (here used as an inlet mean for the washing solution) to inlet mean (2) (here used as an outlet means for this washing solution). An aminopropyl solid phase material (NH₂ layer) was reliably adopted for use as the first clean-up layer in this column.

The second set of layers to which the analytes bind uses an immunoaffinity principle based on an antibody-analyte interaction system.

In the present method the solid phase clean-up and the immunoassaying of the sample are carried out simultaneously in the same column.

A column as generally described in Example 5 was prepared and the method was optimized, for instance, to more specific requirements for detecting OA and AfB1 in a sample of spices. Anti-Ochratoxin A antibodies, Anti-Aflatoxin B1 antibodies, HRP-OA conjugate and HRP-AfB1 conjugate were obtained from the Institute of Animal Sciences, Agricultural Biotechnology, (Gödöllö, Hungary). The anti-OA and the anti-AfB1 were coupled with the CNBr-activated gel (Pharmacia Biotech, Sweden) (for preparing the detection layer) and diluted with blocked gel as described in Example 5. Aminopropyl (solid phase material for preparing the clean up layer) was obtained from J. T. Baker (Belgium) while ColorBurst® Blue was obtained from ALerCHECK Inc. (USA).

Spices (2.5 g) were extracted with 7.5 ml 50% Methanol/3% aqueous NaHCO₃ (80/20, v/v) by shaking for 15 minutes at 200 rpm with an orbital shaker.

The choice of the extraction solvent was made after the following experiment:

For mycotoxin extraction MeOH-water solutions are usually used, for selection of optimal extraction condition methanol-water (pH 5.5) and methanol-NaHCO3 (pH 9.0) solutions of mycotoxins were tested. For stability of detection immunolayers in the clean-up tandem immunoassay columns PBS was used. To control possible PBS effects on the clean-up layer dry NH₂ layer and NH₂ layer kept in contact with PBS for 1 hour, 1 day and 10 days were tested.

It was shown that recovery of both OTA and AfB1 from the NH₂ clean-up layer depended only on clean-up conditions, and not on mycotoxin concentration in the range 2 ng ml⁻¹-6 ng ml⁻¹. Presence of NaHCO₃ dramatically influenced the OTA recovery from the NH₂ clean-up layer (FIG. 2). Application of MeOH/3% NaHCO₃ water solution (25/75, v/v) with pH 9.0 gave very high OA recoveries ranging between 87-93%, but application of MeOH/water (25/75, v/v) with pH 5.5 resulted in very weak OA recoveries (8-12%). In the case of AfB1 no pH effect was obtained, but recoveries from clean-up layer were slightly lower (77-90%) than for OA. MeOH/3% NaHCO₃ water solution was therefore chosen for extraction and extract dilution before clean-up tandem multi immunoassay column application.

For completeness of AfB1 and OA extraction, addition of sodium chloride can be useful but it was here not necessary. The sample was filtered through an Ederol filter and 0.5 ml of extract were diluted with 1 ml with 3% aqueous NaHCO₃. This diluted sample was drawn into the tube (1) through the inlet mean (2) using the plunger (11) of a 20 ml syringe. After that the solution passed through all layers (4, 5 and 14), pressure was applied on the plunger (11) to flow the solution through the column again toward the inlet mean (2) which acted here therefore also as an outlet means. Subsequently, the column was washed with 3 ml PBS-Tween 0.05%. The next step was the application of a mixture of OA-HRP conjugate and AfB1-HRP conjugate using a micropipette on the top grid (15) and removing of the excess conjugate with 3 ml PBS-Tween 0.05%. A 50 μl volume of ColorBurst® Blue was added with a micropipette.

### Example 7

### Clean-up Tandem Immunoassay Optimization

For column preparation two different immunogels were used—with coupled anti-AfB1 and anti-OA primary mouse antibodies.

As analytical signal for mycotoxin determination, the colour development of the detection immunolayers was used. So as to obtain reproducible multiassay results in this format, it is useful to have an equal colour intensity for both detection immunolayers and the same time for colour development. Primary parameters influencing colour intensity and time of its development are concentrations of monoclonal primary mouse antibodies and conjugates. Increasing of concentrations of antibodies and conjugate led to more intense developed colour in shorter time. But at the same time the sensitivity of assay decreased. Volume of the extract is also an important parameter. Increasing the extract volume results in higher sensitivity, but the possible matrix effects become stronger. Besides for field test development minimization of the solution volume (especially for organic solution) is important. Here 0.5 ml of extract diluted with 1.0 ml of NaHCO₃ (3%) water solution were used.

Analysis procedures were developed to obtain cut-off levels for AfB1 at 5 μg kg⁻¹ and for OA at 10 μg kg⁻¹. The optimal concentrations of monoclonal antibody solution were 10 μg ml⁻¹ (dilution 1/100) for monoclonal anti-OA antibodies and 2.5 μg ml⁻¹ (dilution 1/400) for monoclonal anti-AfB1 antibodies. The detection time was chosen as 5 min after chromogen substrate application, and was obtained with conjugate dilutions of 1/100 both for AfB1-HRP and OA-HRP.

For optimization of reagent volumes (conjugate and chromogen substrate solutions) columns with the clean-up layer and two identical detection immunolayers were prepared (both for AfB1 or both for OA detection). Volumes of 50, 100, 150 and 200 μl were tested, and it was shown that 100 μl of conjugate and chromogen substrate solutions were enough to obtain an equal time and intensity of developed colour for the two detection immunolayers. So, a volume of 100 μl was used for all following experiments.

For simultaneous AfB1 and OA determination a set of mycotoxin concentrations was used for validation purposes. As mentioned before, the level of 5 μg kg⁻¹ was legislated for AfB1 in spices and therefore was chosen as cut-off level. A set of samples spiked with 0, 2.0, 3.5, 5.0 μg AfB1 kg¹ was used. According to a presumptive legal limit for OA in spices, the cut-off level was set at 10 μg kg⁻¹. A set of samples spiked with 0, 4.0, 7.0, 10 μg OA kg⁻¹ was used. Samples of chilli, nutmeg, black pepper and ginger fortified with AfB1 and OA were used. The intensity of the developed colours decreased with increasing concentrations of both mycotoxins. No blue colour developed at concentrations of 5 μg kg⁻¹ for AfB1 and 10 μg kg⁻¹ for OA.

### Example 8

### AfB1 and OA Simultaneous Detection in Spice Samples

Thirty nine samples of spices were screened with the clean-up tandem multi immunoassay column. An absence of developed color was interpreted as a positive result, development of blue color shows a negative result. All samples were also analyzed with a clean-up tandem immunoassay column with only one detection layer (for OA or AfB1 only). The same results were obtained as for the column with two detection immunolayers. So we can conclude that neither immunolayers nor OA-HRP and AfB1-HRP conjugates interfered with each other during real sample analysis. This could give an assurance that the proposed approach could be useful for determination of not only two, but more different analytes in one clean-up tandem immunoassay column.

None of seven nutmeg, five black pepper, five white pepper and five ginger samples gave positive results neither for OA nor for AfB1. Totally 35% and 29% of Capsicum ssp. spices were contaminated with OA and AfB1, respectively. In 5 samples both AfB1 and OA and in one red pepper sample only OA was detected higher than the maximum level.

TABLE 1
Toxicants, contaminants and matrices in which they occur matched with their
antibodies and companies they can be obtained from (not exhaustive).

| Contaminants/ | | | |
|---|---|---|---|
| Toxicants | Matrices | Antibodies | Companies |
| Mycotoxins | | | |
| Aflatoxin M₁, M₂, B₁, | Milk, Cheese, nuts, | Anti-AFM₁, M₂, B₁, | Sigma & ICN, IASABC |
| B₂, G₁, G₂ | Beer, cereals | B₂, G₁ and G₂ | |
| | Coffee, feed | | |
| Ochratoxin A | Beer, cereals, | Anti-OA | IASABC |
| | grape juice, wine | | |
| T-2 | Beer, cereals | Anti-T-2 | IASABC |
| Roquefortine | Cheese | Anti-roquefortine | |
| Deoxynivalenol | Cereals, beer | Anti-DON | ICN, Sigma & IASABC |
| Fumonisins | Beer, cereals | Anti-FB₁, FB₂ | ICN, Sigma, Calbiochem |
| Zearalenone | Beer, cereals, feed | Anti-Zea | ICN, Sigma & IASABC |
| Patulin | Apple juice, wine | Anti-patulin | ICN, Calbiochem |
| Hormones | | | |
| Progesterone | Milk | Anti-progesterone | Calbiochem, Sigma & ICN |
| Testosterone | Milk | Anti-testosterone | Sigma & ICN |
| Steroids | Urine | Anti-steroid | Sigma |
| β-agonists | Urine | | |
| Growth hormones | Urine, Blood | | |
| Pesticides | | | |
| Nitrophenols | Water | | |
| Organochlorine | Water | | |
| Atrazine (herbicides) | Water | Anti-atrazine | Millipore |
| Alachlor | Water | Anti-alachlor | Millipore |
| Triazines | Water | | |
| Acetamides | Water | | |
| 2,2-bis(4- | Urine | | |
| chlorophenyl) acetic | | | |
| acid (DDA) for DDT | | | |
| 1-naphthol (Carbory) | Urine | | |
| Antibiotics | | | |
| Chloramphenicol | Milk, blood, urine | Anti- | Sigma |
| | | chloramphenicol | |
| Cephalexin (CEX) | Milk | | |

IASABC—Institute of Animal Science, Agricultural Biotechnology Center, Gödöllö, Hungary

TABLE 2
Evaluation results of six different solid phases for
the effective removal of matrix interferences.

| | | Matrix interference adsorption | | |
|---|---|---|---|---|
| | | Interfering | Flow-through | |
| Type of | Brown color | peak adsorption | Internal | Sample |
| solid phase | adsorption | (HPLC) | control | spot |
| SAX | Strong | No adsorption, | Present | None |
| | | peak appeared | | |
| PAS | Strong | adsorbed, peak | Present | None |
| | | disappeared | | |
| CN | None | No adsorption, | None | None |
| | | peak appeared | | |
| Octadecyl | None | No adsorption, | None | None |
| | | peak appeared | | |
| Diol | None | No adsorption, | None | None |
| | | peak appeared | | |
| AMINOPROPYL | Strong | adsorbed, peak | Present | Present |
| | | disappeared | | |

TABLE 3
Effect of NaHCO₃ concentration on the adsorption of matrix
interference peak by the AMINOPROPYL solid phase material.

| | Interfering matrix peak area | |
|---|---|---|
| Extraction Solution | (retention time at 10 min.) | |
| 50% methanol/50% water | 963.5 | peak area units (pau) |
| 50% methanol/1.5% | 479.45 | pau |
| aqueous NaHCO₃ (1/1) | | |
| 50% methanol/3% | 396.25 | pau |
| aqueous NaHCO₃ (1/1) | | |
| 50% methanol/4% | 127.59 | pau |
| aqueous NaHCO₃ (1/1) | | |
| 50% methanol/6% | | — |
| aqueous NaHCO₃ (1/1) | | |
| 50% methanol/8% | | — |
| aqueous NaHCO₃ (1/1) | | |

TABLE 4
Recoveries of OA by solid phase extraction
using AMINOPROPYL material for clean-up.

| OA concentration spiked into samples | Recovery (%) |
|---|---|
| (ng · g⁻¹) | (n = 5) |
| 2.5 | 81 ± 2 |
| 5 | 74 ± 1 |
| 10 | 84 ± 1 |
| 20 | 72 ± 1 |
| 40 | 74 ± 1 |

TABLE 5
Calculated partition coefficient values for the dispersion
of OA between the aminopropyl solid phase and the methanol/5%
aqueous NaHCO₃ (1/1, vol/vol) mobile phase.

| OA | | MeOH/5% | |
|---|---|---|---|
| Concentration | Aminopropyl | NaHCO₃ (1/1, | Partition coefficient |
| (ng · g⁻¹) | (NH₂) | vol/vol) | (K_{d} = solid/mobile) |
| 20 | 11.42 | 8.58 | 1.33 |
| 40 | 21.22 | 18.78 | 1.13 |
| 80 | 25.72 | 54.29 | 0.47 |
| 160 | 79.88 | 80.13 | 0.997 |

TABLE 6
Between day repeatabilities of the column-based
tandem solid-phase clean-up enzyme immunoassay
for roasted coffee spiked with OA standard.

| | Ochratoxin A concentration (ng · g⁻¹) | | | | |
|---|---|---|---|---|---|
| Day | 0 | 2 | 4 | 6 | 8 |
| 1 | −−− | −−− | +++ | +++ | +++ |
| 2 | −−− | −−+ | +++ | +++ | +++ |
| 3 | −−− | −−− | +++ | +++ | +++ |
| 4 | −−− | −−− | +++ | +++ | +++ |
| 5 | −−− | −−− | +++ | +++ | +++ |

−−− = intense blue (negative);
−−+ = less intense blue (slightly positive);
+++ = no color (very positive)

1. A method for detecting the presence or absence of one or more analytes in a fluid or semi-fluid sample containing an interfering fraction, said method comprising the steps of:
(a) applying said sample in a flow-through motion onto an adsorbent medium comprising at least two layers superposed such as to define a first set of at least one layer and a second set of at least one layer in which said first set of layers is capable of actively adsorbing at least a part of said interfering fraction of said sample without retaining specifically said one or more analytes and whereby said second set of layers is capable of specifically retaining said one or more analytes.
(b) detecting the presence or absence of said one or more analytes specifically retained in said second set of layers.

2. The method according to claim 1, wherein said first set of layers comprises one layer.

3. The method according to claim 1, wherein said first set of layers comprises a derivatized surface.

4. The method according to claim 1, wherein said second set of layers comprises at least two layers, each layer capable of recognizing one of said one or more analytes.

5. The method according to claim 1, wherein at least one of said second set of layers comprises an analyte-receptor.

6. The method according to claim 1, said method comprising the steps of:
(a) applying said sample to a device comprising:
(i) a transparent housing,
(ii) an inlet means for said sample,
(iii) an outlet means for said sample, and
(iv) an adsorbent medium comprising at least two layers superposed such as to define a first set of layers and a second set of layers in which said first set of layers is capable of actively adsorbing at least a part of said interfering fraction of said sample without retaining specifically said one or more analytes and whereby said second set of layers is capable of specifically retaining said one or more analytes,
whereby said sample is applied to said device via said inlet means; and
(b) detecting the presence or absence of said one or more analytes retained in said second set of layers.

7. The method according to claim 5, wherein said analyte-receptor is a protein or peptide selected from the group consisting of: a receptor, a ligand, a substrate, an enzyme, an antibody, and an antigen.

8. The method according to claim 5, in which said analyte-receptor is an antibody specifically recognizing one of said one or more analytes in said sample.

9. The method according to claim 1, in which said step of detecting of the presence of said one or more analytes retained in said second layer is done visually.

10. The method according to claim 1, further comprising the following steps before step (b):
(a′) washing the adsorbent medium in order to remove possible color interference of the second set of layers, and
(a″) applying a predetermined amount of one or more binder molecules onto said adsorbent medium, each of said one or more binder molecules being capable of being retained specifically by one of said second set of layers.

11. The method according to claim 10, wherein said one or more binder molecules are labeled with an enzyme or a bioluminescent, chemiluminescent, phosphorescent or fluorescent molecule.

12. The method according to claim 10, wherein said one or binder molecules is labeled with an enzyme and said method further comprises the following step after step (a″) and before step (b): applying a substrate onto said adsorbent medium, said substrate being capable of reacting with said one or more labeled binder molecules and being capable of generating a detectable signal.

13. The method of claim 12, further comprising the step of washing said adsorbent medium in order to remove all unbound binder molecules from the second set of layers before applying said substrate onto said absorbent medium.

14. The method of claim 10, wherein said predetermined amount of said one or more binder molecules is a predetermined amount of one or more labeled analytes able to provide detection of the absence or presence of the corresponding analyte of interest in the second set of layers.

15. The method according to claim 14 further comprising after step (a″) and before step (b) applying a substrate onto said adsorbent medium, said substrate being capable of reacting with said one or more labeled analyte molecules and being capable of generating a detectable signal.

16. The method according to claim 1, further comprising the step of pre-treating said sample by dissolving or extracting said sample with a specific solvent-, wherein the pretreatment extracts, concentrates or dissolves said one or more analytes.

17. The method according to claim 16, which further comprises diluting said specific solvent prior to flowing said sample through said adsorbent medium.

18. The method according to claim 17, wherein said second layer comprises one or more analyte receptors which are proteins and said specific solvent, after dilution, comprises between 0 and 30% of organic solvent and between 70 and 100% of an aqueous solvent.

19. The method according to claim 1, wherein said sample is applied onto said adsorbent medium by means of pressure.

20. The method according to claim 1, wherein said one or more analytes in said sample are selected from the group consisting of toxins, mycotoxins, pesticides, drugs, antibiotics, hormones, and their respective conjugates, metabolites and derivatives.

21. The method according to claim 20, in which one of said one or more analytes in said sample under investigation is ochratoxin A.

22. The method of claim 6, wherein said sample is applied onto said absorbent medium of said device by a pressure means capable of exerting pressure upon said sample to force the transport of the sample from said inlet means to said outlet means.

23. The method of claim 22, wherein said housing of the device consists of a syringe and the pressure means comprises a syringe plunger.

24. The method of claim 3, wherein said first set of layers comprises a solid support material selected from the group consisting of agarose, silica, sepharose or dextrans and wherein at least part of the surface of said solid support material is derivatized to produce a bonded matrix.

25. The method of claim 3, in which said derivatized surface is derivatized with aminopropyl groups.
